(19) United States
(12) Patent Application Publication
(10) Pub. No.: US 2022/0196663 A1
(43) Pub. Date: Jun. 23, 2022

(54) Reagents and Methods for Cancer Detection, Prognosis, and Therapeutic Monitoring

(71) Applicants: SANFORD HEALTH, Sioux Falls, SD (US); INANOVATE, INC., Sioux Falls, SD (US)

(72) Inventors: Kristi EGLAND, Sioux Falls, SD (US); Rick EVANS, Sioux Falls, SD (US); Eagappanath THIRUPPATHI, Sioux Falls, SD (US); David URE, Sioux Falls, SD (US); Matt KOSTURA, Sioux Falls, SD (US); Etienne Z. GNIMPIEBA, Sioux Falls, SD (US)

(21) Appl. No.: 17/611,351

(22) PCT Filed: Jun. 9, 2020
(86) PCT No.: PCT/US2020/036744

§ 371 Date: Nov. 15, 2021

Related U.S. Application Data
(60) Provisional application No. 62/859,264, filed on Jun. 10, 2019.

(51) Int. Cl. G01N 33/574

(52) U.S. Cl. CPC G01N 33/57415; G01N 2333/8125

(57) ABSTRACT

Methods and related reagents are disclosed for diagnosing cancer, prognosing cancer occurrence or recurrence, and/or monitoring cancer therapy, involving contacting a bodily fluid sample from a subject at risk of having cancer or cancer recurrence, or from a subject that has been treated for cancer wl fit (a) one or more first antibody detection marker molecules that hind to human autoantibodies against at least one tumor associated antigen (TAA); and (fa) one or more second antibody detection marker molecules that bind to human autoantibodies against at least one extractable nuclear antigen (ENA).

### CROSS-REFERENCE

This application claims priority to U.S. Provisional Patent Application Ser. No. 62/859,264 filed Jun. 10, 2019, incorporated by reference herein in its entirety.

### FEDERAL FUNDING STATEMENT

This invention was made with government support under Grant No. P20GM103548 awarded by the National Institutes of Health and under Grant #1R44CA217447-01 from the National Cancer Institute. The government has certain rights in the invention.

### STATEMENT REGARDING SEQUENCE LISTING

This application contains a Sequence Listing submitted as an electronic text file named “17-689-PCT_Sequence-Listing_ST25.txt”, having a size in bytes of 152 kb, and created on Jun. 4, 2020. The information contained in this electronic file is hereby incorporated by reference in its entirety pursuant to 37 CFR § 1.52(e)(5).

### BACKGROUND

The 5-year survival rate for women diagnosed with local breast cancer (BCa) is 98.6%. Survival declines to 83.8% for regional stage and plummets to 23.3% for distant stage. Only 5% of U.S. women present with metastatic BCa when first diagnosed; however, most BCa-related deaths are due to incurable metastatic disease, not the primary diagnosis. Unfortunately, recurrence of BCa is most often found when patients report symptoms, such as shortness of breath, chronic cough, weight loss or bone pain.

### SUMMARY

In one aspect, the disclosure provides methods for diagnosing cancer, prognosing cancer occurrence or recurrence, and/or monitoring cancer therapy, comprising contacting a bodily fluid sample from a subject at risk of having cancer or cancer recurrence, or from a subject that has been treated for cancer with;

(a) one or more first antibody detection marker molecules that bind to human autoantibodies against at least one tumor associated antigen (TAA); and

(b) one or more second antibody detection marker molecules that bind to human autoantibodies against at least one extractable nuclear antigen (ENA);

wherein the presence of autoantibodies against one or more TAA and one or more ENA indicate a likelihood of the subject having cancer or cancer recurrence, and/or indicates efficacy of cancer therapy.

In one embodiment, the at least one ENA is selected from the group consisting of CENPA, Jo-1-U1-snRNP subunit 70 kD, U1-snRNP subunit A, U1-snRNP subunit C, Sm subunit B, Sm subunit B′, Sm subunit D, Sm subunit E, Sm subunit F, Sm subunit G, Scl70, Sm, SS-A/Ro (52 kDa), SS-B/La, CENPB, U1-snRNP, PM/Scl100, SS-A/Ro (60 kDa), Ku, and PM/Scl75. In another embodiment, the at least one TAA comprises at least one breast cancer TAA. In a further embodiment, the at least one TAA is selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, GRN, and GRP78. In one embodiment, the second antibody detection marker molecules that bind to human autoantibodies against at least 2, 3, 4, 5, 6, 7, 8, 9, 10, or more ENAs. In another embodiment, the first antibody detection marker molecules that bind to human autoantibodies against at least 2, 3, 4, 5, 6, 7, 8, 9, 10, or more TAAs. In various further embodiments, the one or more ENA comprises 1, 2, 3, or all 4 of SS-A/Ro (52 kDa), SS-B/La, CENPB, and Ku; the one or more TAA comprises 1, 2, 3, 4, 5, 6, 7, 8, 9, 10, or all 11 of ANGPTL4, CALD1, CD320, CDH3, DKK1, LRRC15, MUC1, SPINT2, SPON2, SSR2, and GRP78; the one or more TAA comprises 1, 2, 3, 4, 5, 6, or all 7 of DKK1, ANGPTL4, MUC1, GRP78, GFRA1, A1AT, and GRN; or the one or more ENA comprises 1, 2, or all 3 of SS-A/Ro, SS-B/LA, and Ku.

In one embodiment, the subject is at risk of having breast cancer or breast cancer recurrence. In another embodiment, the subject is being treated for breast cancer. In another embodiment, the subject has been diagnosed as having breast cancer by other techniques, including but not limited to mammography, wherein the method is used to identify false positive breast cancer diagnoses. In a further embodiment, the one or more first antibody detection marker molecules comprise or consist of the TAA, an antigenic fragment thereof, or a native extracellular domain and/or native secreted version of the TAA or antigenic fragment thereof; and the one or more second antibody detection marker molecules comprise or consist of the ENA or antigenic fragment thereof, or a native extracellular domains and/or native secreted version of the ENA or antigenic fragment thereof. In various other embodiments, the antibody detection marker molecules are detectably labeled; the antibody detection marker molecules are immobilized on a surface; the contacting comprises use of Longitudinal Assay Screening, wherein all target biomarkers may be detected and quantitated within a single test and dilution; the bodily fluid sample comprises a blood sample or serum sample from the subject; the method identifies the subject as likely to have cancer or cancer recurrence, and wherein the method further comprises treating the subject with an amount of a therapeutic sufficient to treat the cancer or cancer recurrence; the treatment comprises one or more of chemotherapy, hormonal therapy, radiation therapy, and/or surgery to remove the tumor.

In another aspect, the disclosure provides antibody detection markers composition comprising

(a) one or more first antibody detection marker molecules that bind to human autoantibodies against at least one tumor associated antigen (TAA); and

(b) one or more second antibody detection marker molecules that bind to human autoantibodies against at least one extractable nuclear antigen (ENA). In one embodiment, the one or more ENA is selected from the group consisting of CENPA, Jo-1, U1-snRNP subunit 70 kD, U1-snRNP subunit A, U1-snRNP subunit C, Sm subunit B, Sm subunit B′, Sm subunit D, Sm subunit E, Sm subunit F, Sm subunit G, Scl70, Sm, SS-A/Ro (52 kDa), SS-B/La, CENPB, U1-snRNP, PM/Scl100, SS-A/Ro (60 kDa), Ku, and PM/Scl75. In another embodiment, the at least one TAA comprises at least one breast cancer TAA. In a further embodiment, the at least one TAA is selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, and GRP78. In one embodiment, the second antibody detection marker molecules bind to human autoantibodies against at least 2, 3, 4, 5, 6, 7, 8, 9, 10, or more ENAs. In another embodiment the first antibody detection marker molecules bind to human autoantibodies against at least 2, 3, 4, 5, 6, 7, 8, 9, 10, or more TAAs. In various further embodiments, the at least one ENA includes 1, 2, 3, or all 4 of SS-A/Ro (52 kDa), SS-B/La, CENPB, and Ku; and/or the at least one TAA includes 1, 2, 3, 4, 5, 6, 7, 8, 9, 10, or all 11 of ANGPTL4, CALD1, CD320, CDH3, DKK1, LRRC15, MUC1, SPINT2, SPON2, SSR2, and GRP78. In one embodiment, the one or more first antibody detection marker molecules comprise or consist of the TAA, an antigenic fragment thereof, or a native extracellular domain and/or native secreted version of the TAA or antigenic fragment thereof; and the one or more second antibody detection marker molecules comprise or consist of the ENA or antigenic fragment thereof, or a native extracellular domains and/or native secreted version of the ENA or antigenic fragment thereof. In another embodiment, the composition consists of between 2-10,000 antibody detection marker molecules in total. In a further embodiment, the composition consists of between 2 and 1000 antibody detection marker molecules in total.

### DESCRIPTION OF THE FIGURES

FIG. 1(A-D). ANA staining analysis of sera from breast cancer patients. Representative photos of ANA staining patterns using 1:40 diluted serum from 6 patients against HEp-2 cells. A. Centromeric; B. Homogeneous; C. Nucleolar; D. Mitochondrial; E. Speckled; F. Negative staining serum sample from cancer patient.

FIG. 2. Positive ENA breast cancer patients. ENA assays were performed using Luminex multiplex assays on serum from 200 cancer patients. The number of positive patients against ENA biomarkers are shown and divided into 2 categories, diagnosed autoimmune condition (left) or no autoimmune disease (right).

FIG. 3. Spot quality QC. All spots are the same diameter with no shape deformation or comet tailing. The results easily passed our targeted print/array QC metric of <10% spot size variability.

FIG. 4. Final layout of 40-plex panel on the Bio-ID cartridges with replicates and controls. Print concentrations for all breast cancer antigens and rFc control are 0.4 mg/ml, excepting LAMC2, which is printed at 0.3 mg/ml. All ENA antigens are printed at 0.25 mg/mL, and all other controls are printed at 0.2 mg/ml.

FIG. 5(A-C). (A) AMLI Consensus Reference Panel Sample for Jo-1 (qualified to contain high concentration of Jo-1). Jo-1 is a specific autoantibody marking inflammatory myopathy and no other systemic autoimmune disease. Jo-1 frequency in the general, healthy population is <5% and titers are typically low. Reference sera shows highly specific and significant signal at 1:8000 dilution. Dilution linearity is also clearly demonstrated. (B) Dilution data on Jo-1 shown as raw signal intensity plots from the Bio-ID. Signal intensity increases over time (cycle number) in a concentration dependent manner (dilution linearity). (C) Reference sera qualified for: CENPA, CENPB, Jo-1, Pm/Scl75, SSA(Ro52), SSA(Ro60), SSB(La), Scl70 and Sm were combined at a mix of dilutions, and processed in a single run. All markers are easily detectible within just 5 cycles on the Bio-ID (14 cycles are used for patient sample processing, further expanding the detectible range to build in significant redundancy on required detection range).

FIG. 6. Illustrates the ROC curve and resultant Area Under Curve (AUC) from a multivariable conditional logistic regression analysis of the biomarker data set. This illustrates an AUC of 0.82, with a Specificity of 81% at a Sensitivity of 72%. The biomarkers identified as having the highest predictive value for discriminating healthy from cancerous samples in a single test using this model are listed in Table 4.

### DETAILED DESCRIPTION

In a first aspect, the disclosure provides methods for diagnosing cancer, prognosing cancer occurrence or recurrence, and/or monitoring cancer therapy, comprising contacting a bodily fluid sample from a subject at risk of having cancer or cancer recurrence, or from a subject that has been treated for cancer with;

(a) one or more first antibody detection marker molecules that bind to human autoantibodies against at least one tumor associated antigen (TAA); and

(b) one or more second antibody detection marker molecules that bind to human autoantibodies against at least one extractable nuclear antigen (ENA);

wherein the presence of autoantibodies against one or more TAA and one or more ENA indicate a likelihood of the subject having cancer or cancer recurrence, and/or indicates efficacy of cancer therapy.

As described in the examples, the inventors have surprisingly discovered that the methods of the disclosure provide significant improvements over prior methods for diagnosing cancer, prognosing cancer occurrence or recurrence, and/or monitoring cancer therapy.

Any suitable antibody detection marker molecules that bind to human autoantibodies against at least one ENA may be used. In a specific embodiment, the at least one ENA is selected from the group consisting of CENPA, Jo-1, U1-snRNP subunit 70 kD, U1-snRNP subunit A, U1-snRNP subunit C, Sm subunit B, Sm subunit B′, Sm subunit D, Sm subunit E, Sm subunit F, Sm subunit G (referred to cumulatively herein as “Sm”), Scl70, Sm, SS-A/Ro (52 kDa; 60 kDa; or both), SS-B/La, CENPB, U1-snRNP, PM/Scl100, Ku, and PM/Scl75. The full name of each ENA is as follows:
CENPA (Centromere protein A)
Jo-1 (Histidyl tRNA synthetase)
U1-snRNP subunit 70 kD, A and C
Scl70 (DNA topoisomerase I)
Sm-subunits B, B′, D, E, F and G
SS-A/Ro, 52 kDa (Tripartite motif-containing 21)
SS-B/La (Small RNA biding exonuclease protection factor La)
CENPB (Centromere protein B)
PM/Scl100 (Exosome component 10)
SS-A/Ro, 60 kDa (Y RNA binding protein)
Ku is composed of two subunits, names and sequences listed separately
   Ku70 (X-ray repair cross complementing 6)
   Ku80 (X-ray repair cross complementing 5)
Pm/Scl75 (Exosome component 9)

Full-length amino acid sequences of each ENA are as follows; these are the ENA antibody detection molecules used in the examples. As will be understood by those of skill in the art, the full-length protein target may be used or other truncated versions of the protein, as well as any other suitable ENA antibody detection molecules:

CENPA sequence
(SEQ ID NO: 1)
MGPRRRSRKPEAPRRRSPSPTPTPGPSRRGPSLGASSHQHSRRRQGWLKEIRKLQKSTHLLIRKLPFSRLAREIC
VKFTRGVDFNWQAQALLALQEAAEAFLVHLFEDAYLLTLHAGRVILFPKDVQLARRIRGLEEGLG
Jo-1 sequence
(SEQ ID NO: 2)
MAERAALEELVKLQGERVRGLKQQKASAELIEEEVAKLLKLKAQLGPDESKQKFVLKTPKGTRDYSPRQMAVREK
VFDVIIRCFKRHGAEVIDTPVFELKETLMGKYGEDSKLIYDLKDQGGELLSLRYDLTVPFARYLAMNKLINIKRY
HIAKVYRRDNPAMTRGRYREFYQCDFDIAGNFDPMIPDAECLKIMCEILSSLQIGDFLVKVNDRRILDGMFAICG
VSDSKFRTICSSVDKLDKVSWEEVKNEMVGEKGLAPEVADRIGDYVQQHGGVSLVEQLLQDPKLSQNKQALEGLG
DLKLLFEYLTLFGIDDKISFDLSLARGLDYYTGVIYEAVLLQTPAQAGEEPLGVGSVAAGGRYDGLVGMFDPKGR
KVPCVGLSIGVERIFSIVEQRLEALEEKIRTTETQVLVASAQKKLLEERLKLVSELWDAGIKAELLYKKNPKLLN
QLQYCEEAGIPLVAIIGEQELKDGVIKLRSVTSREEVDVRREDLVEEIKRRTGQPLCIC
RNP subunits
U1-suRNP 70Ksequence
(SEQ ID NO: 3)
MTQFLPPNLLALFAPRDPIPYLPPLEKLPHEKHHNQPYCGIAPYIREFEDPRDAPPPTRAETREERMERKRREKI
ERRQQEVETELKMWDPHNDPNAQGDAFKTLFVARVNYDTTESKLRREFEVYGPIKRIHMVYSKRSGKPRGYAFIE
YEHERDMHSAYKHADGKKIDGRRVLVDVERGRTVKGWRPRRLGGGLGGTRRGGADVNIRHSGRDDTSRYDERPGP
SPLPHRDRDRDRERERRERSRERDKERERRRSRSRDRRRRSRSRDKEERRRSRERSKDKDRDRKRRSSRSRERAR
RERERKEELRGGGGDMAEPSEAGDAPPDDGPPGELGPDGPDGPEEKGRDRDRERRRSHRSERERRRDRDRDRDRD
REHKRGERGSERGRDEARGGGGGQDNGLEGLGNDSRDMYMESEGGDGYLAPENGYLMEAAPE
SNRPA - subunit A
(SEQ ID NO: 4)
MAVPETRPNHTIYINNLNEKIKKDELKKSLYAIFSQFGQILDILVSRSLKMRGQAFVIFKEVSSATNALRSMQGF
PFYDKPMRIQYAKTDSDIIAKMKGTFVERDRKREKRKPKSQETPATKKAVQGGGATPVVGAVQGPVPGMPPMTQA
PRIMHHMPGQPPYMPPPGMIPPPGLAPGQIPPGAMPPQQLMPGQMPPAQPLSENPPNHILFLTNLPEETNELMLS
MLFNQFPGFKEVRLVPGRHDIAFVEFDNEVQAGAARDALQGFKITQNNAMKISFAKK
SNRPC - subunit C
(SEQ ID NO: 5)
MPKFYCDYCDTYLTHDSPSVRKTHCSGRKHKENVKDYYQKWMEEQAQSLIDKITAAFQQGKIPPTPFSAPPPAGA
MIPPPPSLPGPPRPGMMPAPHMGGPPMMPMMGPPPPGMMPVGPAPGMRPPMGGHMPMMPGPPMMRPPARPMMVPT
RPGMTRPDR
Scl70 sequence
(SEQ ID NO: 6)
MSGDHLHNDSQIEADFRLNDSHKHKDKHKDREHRHKEHKKEKDREKSKHSNSEHKDSEKKHKEKEKTKHKDGSSE
KHKDKHKDRDKEKRKEEKVRASGDAKIKKEKENGFSSPPQIKDEPEDDGYFVPPKEDIKPLKRPRDEDDADYKPK
KIKTEDIKKEKKRKLEEEEDGKLKKPKNKDKDKKVPEPDNKKKKPKKEEEQKWKWWEEERYPEGIKWKFLEHKGP
VFAPPYEPLPENVKFYYDGKVMKLSPKAEEVATFFAKMLDHEYTTKEIFRKNFFKDWRKEMTNEEKNIITNLSKC
DFTQMSQYFKAQTEARKQMSKEEKLKIKEENEKLLKEYGFCIMDNHKERIANFKIEPPGLERGRGNHPKMGMLKR
RIMPEDIIINCSKDAKVPSPPPGHKWKEVRHDNKVTWLVSWTENIQGSIKYIMLNPSSRIKGEKDWQKYETARRL
KKCVDKIRNQYREDWKSKEMKVRQRAVALYFIDKLALRAGNEKEEGETADTVGCCSLRVEHINLHPELDGQEYVV
EFDFLGKDSIRYYNKVPVEKRVFKNLQLFMENKQPEDDLFDRLNIGILNKHLQDLMEGLTAKVERTYNASITLQQ
QLKELTAPDENIPAKILSYNRANRAVAILCNHQRAPPKTFEKSMMNLQTKIDAKKEQLADARRDLKSAKADAKVM
KDAKIKKVVESKKKAVQRLEEQLMKLEVQATDREENKQIALGTSKLNYLDPRITVAWCKKWGVPIEKIYNKTQRE
KFAWAIDMADEDYEF
Smith antigen subunits
SNRPB - subunit B
(SEQ ID NO: 7)
MTVGKSSKMLQHIDYRMRCILQDGRIFIGTFKAFDKHMNLILCDCDEFRKIKPKNSKQAEREEKRVLGLVLLRGE
NLVSMTVEGPPPKDIGIARVPLAGAAGGPGIGRAAGRGIPAGVPMPQAPAGLAGPVRGVGGPSQQVMTPQGRGTV
AAAAAAATASIAGAPTQYPPGRGGPPPPMGRGAPPPGMMGPPPGMRPPMGPPMGIPPGRGIPMGMPPPGMRPPPP
GMRGLL
SNRPB1 - subunit B′
(SEQ ID NO: 8)
MTVGKSSKMLQHIDYRMRCILQDGRIFIGTFKAFDKHMNLILCDCDEFRKIKPKNSKQAEREEKRVLGLVLLRGE
NLVSMTVEGPPPKDTGIARVPLAGAAGGPGIGRAAGRGIPAGVPMPQAPAGLAGPVRGVGGPSQQVMTPQGRGTV
AAAAAAATASIAGAPTQYPPGRGGPPPPMGRGAPPPGMMGPPPGMRPPMGPPMGIPPGRGTPMGMPPPGMRPPPP
GMRGPPPPGMRPPRP
SNRPD1 - subunit D
(SEQ ID NO: 9)
MKLVRFLMKLSHETVTIELKNGTQVHGTITGVDVSMNTHLKAVKMILKNREPVQLETLSIRGNNIRYFILPDSLP
LDTLLVDVEPKVKSKKREAVAGRGRGRGRGRGRGRGRGRGGPRR
SNRPE - subunit E
(SEQ ID NO: 10)
MAYRGQGQKVQKVMVQPINLIFRYLQNRSRIQVWLYEQVNMRIEGCIIGFDEYMNLVLDDAEEIHSKIKSRKQLG
RIMLKGDNITLLQSVSN
SNRPF - subunit F
(SEQ ID NO: 11)
MSLPLNPKPFLNGLTGKPVMVKLKWGMEYKGYLVSVDGYMNMQLANTEEYIDGALSGHLGEVLIRCNNVLYIRGV
EEEEEDGEMRE
SNRPG - subunit G
(SEQ ID NO: 12)
MSKAHPPELKKFMDKKLSLKLNGGRHVQGILRGFDPFMNLVIDECVEMATSGQQNNIGMVVIRGNSIIMLEALER
V
SS-A/Ro 52 kDa sequence
(SEQ ID NO: 13)
MASAARLTMMWEEVTCPICLDPFVEPVSIECGHSFCQECISQVGKGGGSVCPVCRQRFLLKNLRPNRQLANMVNN
LKEISQEAREGTQGERCAVHGERLHLFCEKDGKALCWVCAQSRKHRDHAMVPLEEAAQEYQEKLQVALGELRRKQ
ELAEKLEVEIAIKRADWKKIVETQKSRIHAEFVQQKNFLVEEEQRQLQELEKDEREQLRILGEKEAKLAQQSQAL
QELISELDRRCHSSALELLQEVIIVLERSESWNLKDLDITSPELRSVCHVPGLKKMLRICAVHITLDPDTANPWL
ILSEDRRQVRLGDTQQSIPGNEERFDSYPMVLGAQHFHSGKHYWEVDVIGKEAWDLGVCRDSVRRKGHFLLSSKS
GFWTIWLWNKQKYEAGTYPQTPLHLQVPPCQVGIFLDYEAGMVSFYNITDHGSLIYSFSECAFTGPLRPFFSPGF
NDGGKNTAPLTLCPLNIGSQGSTDY
SS-B/La sequence
(SEQ ID NO: 14)
MAENGDNEKMAALEAKICHQIEYYFGDFNLPRDKFLKEQIKLDEGWVPLEIMIKFNRLNRLITDFNVIVEALSKS
KAELMEISEDKTKIRRSPSKPLPEVTDEYKNDVKNRSVYIKGFPIDATLDDIKEWLEDKGQVLNIQMRRILHKAF
KGSIFVVFDSIESAKKFVETPGQKYKETDLLILFKDDYFAKKNEERKQNKVEAKLRAKQEQEAKQKLEEDAEMKS
LEEKIGCLLKFSGDLDDQTCREDLHILFSNHGEIKWIDFVRGAKEGIILFKEKAKEALGKAKDANNGNLQLRNKE
VTWEVLEGEVEKEALKKIIEDQQESLNKWKSKGRRFKGKGKGNKAAQPGSGKGKVQFQGKKIKFASDDEHDEHDE
NGATGPVKRAREETDKEEPASKQQKTENGAGDQ
CENPB sequence
(SEQ ID NO: 15)
MGPKRRQLTFREKSRIIQEVEENPDLRKGEIARRFNIPPSTLSTILKNKRAILASERKYGVASTCRKTNKLSPYD
KLEGLLIAWFQQIRAAGLPVKGIILKEKALRIAEELGMDDFTASNGWLDRFRRRHGVVSCSGVARARARNAAPRT
PAAPASPAAVPSEGSGGSTTGWRAREEQPPSVAEGYASQDVFSATETSLWYDFLPDQAAGLCGGDGRPRQATQRL
SVLLCANADGSEKLPPLVAGKSAKPRAGQAGLPCDYTANSKGGVTTQALAKYLKALDTRMAAESRRVLLLAGRLA
AQSLDTSGLRHVQLAFFPPGTVHPLERGVVQQVKGHYRQAMLLKAMAALEGQDPSGLQLGLTEALHFVAAAWQAV
EPSDIAACFREAGFGGGPNATITTSLKSEGEEEEEEEEEEEEEEGEGEEEEEEGEEEEEEGGEGEELGEEEEVEE
EGDVDSDEEEEEDEESSSEGLEAEDWAQGVVEAGGSFGAYGAQEEAQCPTLHFLEGGEDSDSDSEEEDDEEEDDE
DEDDDDDEEDGDEVPVPSFGEAMAYFAMVKRYLTSFPIDDRVQSHILHLEHDLVHVIRKNHARQAGVRGLGHQS
Pm/Scl100 sequence
(SEQ ID NO: 16)
MAPPSTREPRVLSATSATKSDGEMVLPGFPDADSFVKFALGSVVAVTKASGGLPQFGDEYDFYRSFPGFQAFCET
QGDRLLQCMSRVMQYHGCRSNIKDRSKVIELEDKFDLLVDANDVILERVGILLDEASGVNKNQQPVLPAGLQVPK
TVVSSWNRKAAEYGKKAKSETFRLLHAKNIIRPQLKFREKIDNSNIPFLPKIFIKPNAQKPLPQALSKERRERPQ
DRPEDLDVPPALADFIHQQRTQQVEQDMFAHPYQYELNHFTPADAVLQKPQPQLYRPIEETPCHFISSLDELVEL
NEKLLNCQEFAVDLEHHSYRSFLGLTCLMQISTRTEDFIIDTLELRSDMYILNESLTDPAIVKVFHGADSDIEWL
QKDEGLYVVNMFDTHQAARLLNLGRHSLDHLLKLYCNVDSNKQYQLADWRIRPLPEEMLSYARDDTHYLLYIYDK
MRLEMWERGNGQPVQLQVVWQRSRDICLKKFIKPIFTDESYLELYRKQKKHLNIQQLTAFQLLFAWRDKTARRED
ESYGYVLPNHMMLKIAEELPKEPQGIIACCNPVPPLVRQQINEMHLLIQQAREMPLLKSEVAAGVKKSGPLPSAE
RLENVLFGPHDCSHAPPDGYPIIPTSGSVPVQKQASLFPDEKEDNLLGTICLIATAVITLFNEPSAEDSKKGPLT
VAQKKAQNIMESFENPFRMFLPSLGHRAPVSQAAKFDPSTKIYEISNRWKLAQVQVQKDSKEAVKKKAAEQTAAR
EQAKEACKAAAEQAISVRQQVVLENAAKKRERATSDPRITEQKQEKKRLKISKKPKDPEPPEKEFTPYDYSQSDF
KAFAGNSKSKVSSQFDPNKQTPSGKKCIAAKKIKQSVGNKSMSFPIGKSDRGFRYNWPQR
SS-A/Ro 60 kDa sequence
(SEQ ID NO: 17)
MEESVNQMQPLNEKQIANSQDGYVWQVIDMNRLHRFLCFGSEGGTYYIKEQKLGLENAEALIRLIEDGRGCEVIQ
EIKSFSQEGRITKQEPMLFALAICSQCSDISTKQAAFKAVSEVCRIPTHLFTFIQFKKDLKESMKCGMWGRALRK
AIADWYNEKGGMALALAVIKYKQRNGWSHKDLLRLSHLKPSSEGLAIVIKYITKGWKEVHELYKEKALSVETEKL
LKYLEAVEKVKRIRDELEVIHLIEEHRLVREHLLINHLKSKEVWKALLQEMPLTALLRNLGKMTANSVLEPGNSE
VSLVCEKLCNEKLLKKARIHPFHILIALETYKIGHGLRGKLKWRPDEEILKALDAAFYKTFKIVEPTGKRFLLAV
DVSASMNQRVLGSILNASTVAAAMCMVVIRTEKDSYVVAFSDEMVPCPVTTDMILQQVLMAMSQIPAGGIDCSLP
MIWAQKINTPADVFIVFTDNETFAGGVHPAIALREYRKKMDIPAKLIVCGMTSNGFTIADPDDRALQNTLLNKSF
Ku70 sequence
(SEQ ID NO: 18)
MSGWESYYKTEGDEEAEEEQEENLEASGDYKYSGRDSLIFLVDASKAMFESQSEDELTPFDMSIQCIQSVYISKI
ISSDRDLLAVVFYGTEKDKNSVNFKNIYVLQELDNPGAKRILELDQFKGQQGQKRFQDMMGHGSDYSLSEVLWVC
ANLFSDVQFKMSHKRIMLFTNEDNPHGNDSAKASRARTKAGDLRDTGIFLDLMHLKKPGGFDISLFYRDIISIAE
DEDLRVHFEESSKLEDLLRKVRAKETRKRALSRLKLKLNKDIVISVGIYNLVQKALKPPPIKLYRETNEPVKIKT
RTFNISTGGLLLPSDTKRSQIYGSRQIILEKEETEELKRFDDPGLMLMGFKPLVLLKKHHYLRPSLFVYPEESLV
IGSSTLFSALLIKCLEKEVAALCRYTPRRNIPPYFVALVPQEEELDDQKIQVIPPGFQLVFLPFADDKRKMPFTE
KIMATPEQVGKMKAIVEKLRFTYRSDSFENPVLQQHFRNLEALALDLMEPEQAVDLTLPKVEAMNKRLGSLVDEF
KELVYPPDYNPEGKVTKRKHDNEGSGSKRPKVEYSEEELKTHISKGTLGKFTVPMLKEACRAYGLKSGLKKQELL
EALTKHFQD
Ku80 sequence
(SEQ ID NO: 19)
MVRSGNKAAVVLCMDVGFTMSNSIPGIESPFEQAKKVITMFVQRQVFAENKDEIALVLFGTDGTDNPLSGGDQYQ
NITVHRHLMLPDFDLLEDIESKIQPGSQQADFLDALIVSMDVIQHETIGKKFEKRHIEIFTDLSSRFSKSQLDII
IHSLKKCDISLQFFLPFSLGKEDGSGDRGDGPFRLGGHGPSFPLKGITEQQKEGLEIVKMVMISLEGEDGLDEIY
SFSESLRKLCVFKKIERHSIHWPCRLTIGSNLSIRIAAYKSILQERVKKIWTVVDAKTLKKEDIQKETVYCLNDD
DETEVLKEDIIQGFRYGSDIVPFSKVDEEQMKYKSEGKCFSVLGFCKSSQVQRRFFMGNQVLKVFAARDDEAAAV
ALSSLIHALDDLDMVAIVRYAYDKRANPQVGVAFPHIKHNYECLVYVQLPFMEDLRQYMFSSLKNSKKYAPTEAQ
LNAVDALIDSMSLAKKDEKTDTLEDLFPITKIPNPRFQRLFQCLLHRALHPREPLPPIQQHIWNMLNPPAEVITK
SQIPLSKIKTLFPLIEAKKKDQVTAQEIFQDNHEDGPTAKKLKTEQGGAHFSVSSLAEGSVTSVGSVNPAENFRV
LVKQKKASFEEASNQLINHIEQFLDTNETPYFMKSIDCIRAFREEAIKFSEEQRFNNFLKALQEKVEIKQLNHFW
EIVVQDGITLITKEEASGSSVTAEEAKKFLAPKDKPSGDTAAVFEEGGDVDDLLDMI
Pm/Scl75 sequence
(SEQ ID NO: 20)
MKETPLSNCERRFLLRAIEEKKRLDGRQTYDYRNIRISFGTDYGCCIVELGKTRVLGQVSCELVSPKLNRATEGI
LFFNLELSQMAAPAFEPGRQSDLLVKLNRLMERCLRNSKCIDTESLCVVAGEKVWQIRVDLHLLNHDGNIIDAAS
IAAIVALCHFRRPDVSVQGDEVTLYTPEERDPVPLSIHHMPICVSFAFFQQGTYLLVDPNEREERVMDGLLVIAM
NKHREICTIQSSGGIMLLKDQVLRCSKIAGVKVAEITELILKALENDQKVRKEGGKEGFAESIANQRITAFKMEK
APIDTSDVEEKAEEIIAEAEPPSEVVSTPVLWTPGTAQIGEGVENSWGDLEDSEKEDDEGGGDQAIILDGIKMDT
GVEVSDIGSQELGFHHVGQTGLEFLTSDAPIILSDSEEEEMIILEPDKNPKKIRTQTTSAKQEKAPSKKPVKRRK
KKRAAN

Any suitable antibody detection marker molecules that bind to human autoantibodies against at least one TAA may be used. The TAA may be associated with a tumor type including but not limited to breast cancer, prostate cancer, ovarian cancer, lung or any other epithelial cancer. In one specific embodiment, the at least one TAA comprises at least one breast cancer TAA.

In another specific embodiment, the at least one TAA is selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, GRN, and GRP78. The full name of each TAA is provided below:
A1AT (Alpha-1 antitrypsin)
ANGPTL4 (Angiopoietin-like 4)
LRP10 (LDL Receptor Related Protein 10)
GFRA1 (GDNF Family Receptor Alpha 1)
DKK1 (Dickkopf WNT Signaling Pathway Inhibitor 1)
GRP78 (78 kDa glucose-regulated protein)
GRN (Granulin)
CALD1 (Caldesmon 1)
CD147 (Basigin)
CD320 (CD320 molecule)
CDH3 (Cadherin 3)
CST2 (Cystatin SA)
EPHA2 (EPH receptor A2)
LGALS1 (Galectin 1)
GRN (Granulin precursor)
ERBB2 (Erb-b2 receptor tyrosine kinase 2)
IGFBP2 (Insulin like growth factor binding protein 2)
LAMC2 (Laminin subunit gamma 2)
LRRC15 (Leucine rich repeat containing 15)
MUC1 (Mucin 1)
SPINT2 (Serine peptidase inhibitor, Kunitz type 2)
SPON2 (Spondin 2)
SSR2 (Signal sequence receptor subunit 2)
SUSD2 (Sushi domain containing 2)
XAGE1B (X antigen family member 1B)
BIRC5 (Baculoviral IAP repeat containing 5)
LGALS3 (Galectin 3)
CAPC/LRRC26 (Leucine rich repeat containing 26)

Exemplary amino acid sequence for each TAA is provided below; these are the TAA antibody detection molecules used in the examples. As will be understood by those of skill in the art, the full-length protein target may be used or other truncated versions of the protein, as well as any other suitable TAA antibody detection molecules:

ANGPTL4
(SEQ ID NO: 21)
KSPRFASWDEMNVLAHGLLQLGQGLREHAERTRSQLSALERRLSACGSACQGTEGSTDLPLAPESRVDPEVLHSL
QTQLKAQNSRIQQLFHKVAQQQRHLEKQHLRIQHLQSQFGLLDHKHLDHEVAKPARRKRLPEMAQPVDPAHNVSR
LHRLPRDCQELFQVGERQSGLFEIQPQGSPPFLVNCKMTSDGGWTVIQRRHDGSVDFNRPWEAYKAGEGDPHGEF
WLGLEKVHSITGDRNSRLAVQLRDWDGNAELLQFSVHLGGEDTAYSLQLTAPVAGQLGATTVPPSGLSVPFSTWD
QDHDLRRDKNCAKSLSGGWWFGTCSHSNLNGQYFRSIPQQRQKLKKGIFWKTWRGRYYPLQATTMLIQPMAAEAA
S
DKK1
(SEQ ID NO: 22)
VSATLNSVLNSNAIKNLPPPLGGAAGHPGSAVSAAPGILYPGGNKYQTIDNYQPYPCAEDEECGTDEYCASPTRG
GDAGVQICLACRKRRKRCMRHAMCCPGNYCKNGICVSSDQNHFRGEIEETITESFGNDHSTLDGYSRRTTLSSKM
YHTKGQEGSVCLRSSDCASGLCCARHFWSKICKPVLKEGQVCTKHRRKGSHGLEIFQRCYCGEGLSCRIQKDHHQ
ASNSSRLHTCQRH
GFRA1
(SEQ ID NO: 23)
DRLDCVKASDQCLKEQSCSTKYRTLRQCVAGKETNFSLASGLEAKDECRSAMEALKQKSLYNCRCKRGMKKEKNC
LRIYWSMYQSLQGNDLLEDSPYEPVNSRLSDIERVVPFISDVFQQVEHIPKGNNCLDAAKACNLDDICKKYRSAY
ITPCTTSVSNDVCNRRKCHKALRQFFDKVPAKHSYGMLFCSCRDIACTERRRQTIVPVCSYEEREKPNCLNLQDS
CKTNYICRSRLADFFTNCQPESRSVSSCLKENYADCLLAYSGLIGTVMTPNYIDSSSLSVAPWCDCSNSGNDLEE
CLKFLNFFKDNTCLKNAIQAFGNGSDVTVWQPAFPVQTTTATTTTALRVKNKPLGPAGSENEIPTHVLPPCANLQ
AQKLKSNVSGNTHLCISNGNYEKEGLGASSHITTKSMAAPPSCGLSPLLVLVVTALSTLLSLTETS
GRN
(SEQ ID NO: 24)
TRCPDGQFCPVACCLDPGGASYSCCRPLLDKWPTTLSRHLGGPCQVDAHCSAGHSCIFTVSGTSSCCPFPEAVAC
GDGHHCCPRGFHCSADGRSCFQRSGNNSVGAIQCPDSQFECPDFSTCCVMVDGSWGCCPMPQASCCEDRVHCCPH
GAFCDLVHTRCITPTGTHPLAKKLPAQRTNRAVALSSSVMCPDARSRCPDGSTCCELPSGKYGCCPMPNATCCSD
HLHCCPQDTVCDLIQSKCLSKENATTDLLTKLPAHTVGDVKCDMEVSCPDGYTCCRLQSGAWGCCPFTQAVCCED
HIHCCPAGFTCDTQKGTCEQGPHQVPWMEKAPAHLSLPDPQALKRDVPCDNVSSCPSSDTCCQLTSGEWGCCPIP
EAVCCSDHQHCCPQGYTCVAEGQCQRGSEIVAGLEKMPARRASLSHPRDIGCDQHTSCPVGQTCCPSLGGSWACC
QLPHAVCCEDRQHCCPAGYTCNVKARSCEKEVVSAQPATFLARSPHVGVKDVECGEGHFCHDNQTCCRDNRQGWA
CCPYRQGVCCADRRHCCPAGFRCAARGTKCLRREAPRWDAPLRDPALRQLL
LRP10
(SEQ ID NO: 25)
HPDRIIFPNHACEDPPAVLLEVQGTLQRPLVRDSRTSPANCTWLILGSKEQTVTIRFQKLHLACGSERLTLRSPL
QPLISLCEAPPSPLQLPGGNVTITYSYAGARAPMGQGFLLSYSQDWLMCLQEEFQCLNHRCVSAVQRCDGVDACG
DGSDEAGCSSDPFPGLTPRPVPSLPCNVTLEDFYGVFSSPGYTHLASVSHPQSCHWLLDPHDGRRLAVRFTALDL
GFGDAVHVYDGPGPPESSRLLRSLTHFSNGKAVTVETLSGQAVVSYHTVAWSNGRGFNATYHVRGYCLPWDRPCG
LGSGLGAGEGLGERCYSEAQRCDGSWDCADGTDEEDCPGCPPGHFPCGAAGTSGATACYLPADRCNYQTFCADGA
DERRCRHCQPGNFRCRDEKCVYETWVCDGQPDCADGSDEWDCSYVLPRK
CST2
(SEQ ID NO: 26)
WSPQEEDRIIEGGIYDADLNDERVQRALHFVISEYNKATEDEYYRRLLRVLRAREQIVGGVNYFFDIEVGRTICT
KSQPNLDTCAFHEQPELQKKQLCSFQIYEVPWEDRMSLVNSRCQEA
A1AT
(SEQ ID NO: 27)
EDPQGDAAQKTDTSHHDQDHPTFNKITPNLAEFAFSLYRQLAHQSNSTNIFFSPVSIATAFAMLSLGTKADTHDE
ILEGLNFNLTEIPEAQIHEGFQELLRTLNQPDSQLQLTTGNGLFLSEGLKLVDKFLEDVKKLYHSEAFTVNFGDT
EEAKKQINDYVEKGTQGKIVDLVKELDRDTVFALVNYIFFKGKWERPFEVKDTEEEDFHVDQVTTVKVPMMKRLG
MFNIQHCKKLSSWVLLMKYLGNATAIFFLPDEGKLQHLENELTHDIITKFLENEDRRSASLHLPKLSITGTYDLK
SVLGQLGITKVFSNGADLSGVTEEAPLKLSKAVHKAVLTIDEKGTEAAGAMFLEAIPMSIPPEVKFNKPFVFLMI
EQNTKSPLFMGKVVNPTQK
GAL1
(SEQ ID NO: 28)
LRVRGEVAPDAKSFVLNLGKDSNNLCLHFNPRFNAHGDANTIVCNSKDGGAWGTEQREAVFPFQPGSVAEVCITF
DQANLTVKLPDGYEFKFPNRLNLEAINYMAADGDFKIKCVAFD
MUC1
(SEQ ID NO: 29)
APKPATVVTGSGHASSTPGGEKETSATQRSSVPSSTEKNAFNSSLEDPSTDYYQELQRDISEMFLQIYKQGGFLG
LSNIKFRPGSVVVQLTLAFREGTINVHDVETQFNQYKTEAASRYNLTISDVSVSDVPFPFSAQSGAGVPG
GRP78
(SEQ ID NO: 30)
EEEDKKEDVGTVVGIDLGTTYSCVGVFKNGRVEIIANDQGNRITPSYVAFTPEGERLIGDAAKNQLTSNPENTVF
DAKRLIGRTWNDPSVQQDIKFLPFKVVEKKTKPYIQVDIGGGQTKTFAPEEISAMVLTKMKETAEAYLGKKVTHA
VVTVPAYFNDAQRQATKDAGTIAGLNVMRIINEPTAAAIAYGLDKREGEKNILVFDLGGGTFDVSLLTIDNGVFE
VVATNGDTHLGGEDFDQRVMEHFIKLYKKKTGKDVRKDNRAVQKLRREVEKAKRALSSQHQARIEIESFYEGEDF
SETLTRAKFEELNMDLFRSTMKPVQKVLEDSDLKKSDIDEIVLVGGSTRIPKIQQLVKEFFNGKEPSRGINPDEA
VAYGAAVQAGVLSGDQDTGDLVLLDVCPLTLGIETVGGVMTKLIPRNTVVPTKKSQIFSTASDNQPTVTIKVYEG
ERPLTKDNHLLGTFDLTGIPPAPRGVPQIEVTFEIDVNGILRVTAEDKGTGNKNKITITNDQNRLTPEEIERMVN
DAEKFAEEDKKLKERIDTRNELESYAYSLKNQIGDKEKLGGKLSSEDKETMEKAVEEKIEWLESHQDADIEDFKA
KKKELEEIVQPIISKLYGSAGPPPTGEEDTAEKDEL
EPRA2
(SEQ ID NO: 31)
KEVVLLDFAAAGGELGWLTHPYGKGWDLMQNIMNDMPIYMYSVCNVMSGDQDNWLRTNWVYRGEAERIFIELKFT
VRDCNSFPGGASSCKETFNLYYAESDLDYGTNFQKRLFTKIDTIAPDEITVSSDFEARHVKLNVEERSVGPLTRK
GFYLAFQDIGACVALLSVRVYYKKCPELLQGLAHFPETIAGSDAPSLATVAGTCVDHAVVPPGGEEPRMHCAVDG
EWLVPIGQCLCQAGYEKVEDACQACSPGFFKFEASESPCLECPEHTLPSPEGATSCECEEGFFRAPQDPASMPCT
RPPSAPHYLTAVGMGAKVELRWTPPQDSGGREDIVYSVTCEQCWPESGECGPCEASVRYSEPPHGLTRTSVTVSD
LEPHMNYTFTVEARNGVSGLVTSRSFRTASVSINQTEPPKVRLEGRSTTSLSVSWSIPPPQQSRVWKYEVTYRKK
GDSNSYNVRRTEGFSVTLDDLAPDTTYLVQVQALTQEGQGAGSKVHEFQTLSPEGSGNL
LAMC2
(SEQ ID NO: 32)
TSRREVCDCNGKSRQCIFDRELHRQTGNGFRCLNCNDNTDGIHCEKCKNGFYRHRERDRCLPCNCNSKGSLSARC
DNSGRCSCKPGVTGARCDRCLPGFHMLTDAGCTQDQRLLDSKCDCDPAGIAGPCDAGRCVCKPAVTGERCDRCRS
GYYNLDGGNPEGCTQCFCYGHSASCRSSAEYSVHKITSTFHQDVDGWKAVQRNGSPAKLQWSQRHQDVESSAQRL
DPVYFVAPAKFLGNQQVSYGQSLSFDYRVDRGGRHPSAHDVILEGAGLRITAPLMPLGKTLPCGLTKTYTFRLNE
HPSNNWSPQLSYFEYRRLLRNLTALRIRATYGEYSTGYIDNVTLISARPVSGAPAPWVEQCICPVGYKGQFCQDC
ASGYKRDSARLGPFGTCIPCNCQGGGACDPDTGDCYSGDENPDIECADCPIGFYNDPHDPRSCKPCPCHNGFSCS
VMPETEEVVCNNCPPGVTGARCELCADGYFGDPFGEHGPVRPCQPCQCNNNVDPSASGNCDRLTGRCLKCIHNTA
GIYCDQCKAGYFGDPLAPNPADKCRACNCNPMGSEPVGCRSDGTCVCKPGFGGPNCEHGAFSCPACYNQVKIQMD
QFMQQLQRMEALISKAQGGDGVVPDTELEGRMQQAEQALQDILRDAQISEGASRSLGLQLAKVRSQENSYQSRLD
DLKMTVERVRALGSQYQNRVRDTHRLITQMQLSLAESEASLGNTNIPASDHYVGPNGFKSLAQEATRLAESHVES
ASNMEQLTRETEDYSKQALSLVRKALHEGVGSGSGSPDGAVVQGLVEKLEKTKSLAQQLTREATQAEIEADRSYQ
HSLRLLDSVSRLQGVSDQSFQVEEAKRIKQKADSLSSLVTRHMDEFKRTQKNLGNWKEEAQQLLQNGKSGREKSD
QLLSRANLAKSRAQEALSMGNATFYEVESILKNLREFDLQVDNRKAEAEEAMKRLSYISQKVSDASDKTQQAERA
LGSAAADAQRAKNGAGEALEISSEIEQEIGSLNLEANVTADGALAMEKGLASLKSEMREVEGELERKELEFDTNM
DAVQMVITEAQKVDTRAKNAGVTIQDTLNTLDGLLHLMGM
SPON2
(SEQ ID NO: 33)
QPLGGESICSARAPAKYSITFTGKWSQTAFPKQYPLFRPPAQWSSLLGAAHSSDYSMWRKNQYVSNGLRDFAERG
EAWALMKEIEAAGEALQSVHEVFSAPAVPSGTGQTSAELEVQRRHSLVSFVVRIVPSPDWFVGVDSLDLCDGDRW
REQAALDLYPYDAGTDSGFTFSSPNFATIPQDTVTEITSSSPSHPANSFYYPRLKALPPIARVTLLRLRQSPRAF
IPPAPVLPSRDNEIVDSASVPETPLDCEVSLWSSWGLCGGHCGRLGTKSRTRYVRVQPANNGSPCPELEEEAECV
PDNCV
SSR2
(SEQ ID NO: 34)
EEGARLLASKSLLNRYAVEGRDLTLQYNIYNVGSSAALDVELSDDSFPPEDFGIVSGMLNVKWDRIAPASNVSHT
VVLRPLKAGYFNFTSATITYLAQEDGPVVIGSTSAPGQGGILAQREFDRRFSPH
LRRC15
(SEQ ID NO: 35)
YHGCPSECTCSRASQVECTGARIVAVPTPLPWNAMSLQILNTHITELNESPFLNISALIALRIEKNELSRITPGA
FRNLGSLRYLSLANNKLQVLPIGLFQGLDSLESLLLSSNQLLQIQPAHFSQCSNLKELQLHGNHLEYIPDGAFDH
LVGLTKLNLGKNSLTHISPRVFQHLGNLQVLRLYENRLTDIPMGTFDGLVNLQELALQQNQIGLLSPGLFHNNHN
LQRLYLSNNHISQLPPSVFMQLPQLNRLTLFGNSLKELSPGIFGPMPNLRELWLYDNHISSLPDNVFSNLRQLQV
LILSRNQISFISPGAFNGLTELRELSLHTNALQDLDGNVERMLANLQNISLQNNRLRQLPGNIFANVNGLMAIQL
QNNQLENLPLGIFDHLGKLCELRLYDNPWRCDSDILPLRNWLLLNQPRLGTDTVPVCFSPANVRGQSLIIINVNV
AVPSVHVPEVPSYPETPWYPDTPSYPDTTSVSSTTELTSPVEDYTDLTTIQVTDDRSVWGMTQAQSG
MUC1
(SEQ ID NO: 36)
APKPATVVTGSGHASSTPGGEKETSATQRSSVPSSTEKNAFNSSLEDPSTDYYQELQRDISEMFLQIYKQGGFLG
LSNIKFRPGSVVVQLTLAFREGTINVHDVETQFNQYKTEAASRYNLTISDVSVSDVPFPFSAQSGAGVPG
CD147
(SEQ ID NO: 37)
AAGTVFTTVEDLGSKILLTCSLNDSATEVTGHRWLKGGVVLKEDALPGQKTEFKVDSDDQWGEYSCVFLPEPMGT
ANIQLHGPPRVKAVKSSEHINEGETAMLVCKSESVPPVTDWAWYKITDSEDKALMNGSESRFFVSS
CD320
(SEQ ID NO: 38)
AGPSSGSCPPTKFQCRTSGLCVPLTWRCDRDLDCSDGSDEEECRIEPCTQKGQCPPPPGLPCPCTGVSDCSGGTD
KKLRNCSRLACLAGELRCTLSDDCIPLTWRCDGHPDCPDSSDELGCGTNEILPEGDATTMGPPVTLESVTSLRNA
TTMGPPVTLESVPSVGNATSSSAGDQSGSPTAYG
CDH3
(SEQ ID NO: 39)
EPCRAVFREAEVTLEAGGAEQEPGQALGKVFMGCPGQEPALFSTDNDDFTVRNGETVQERRSLKERNPLKIFPSK
RILRRHKRDWVVAPISVPENGKGPFPQRLNQLKSNKDRDTKIFYSITGPGADSPPEGVFAVEKETGWLLLNKPLD
REEIAKYELFGHAVSENGASVEDPMNISIIVTDQNDHKPKFTQDTFRGSVLEGVLPGTSVMQMTATDEDDAIYTY
NGVVAYSIHSQEPKDPHDLMFTIHRSTGTISVISSGLDREKVPEYTLTIQATDMDGDGSTTTAVAVVEILDANDN
APMFDPQKYEAHVPENAVGHEVQRLTVTDLDAPNSPAWRATYLIMGGDDGDHFTITTHPESNQGILTTRKGLDFE
AKNQHTLYVEVTNEAPFVLKLPTSTATIVVHVEDVNEAPVFVPPSKVVEVQEGIPTGEPVCVYTAEDPDKENQKI
SYRILRDPAGWLAMDPDSGQVTAVGTLDREDEQFVRNNIYEVMVLAMDNGSPPTTGTGTLLLTLIDVNDHGPVPE
PRQITICNQSPVRQVLNITDKDLSPHTSPFQAQLTDDSDIYWTAEVNEEGDTVVLSLKKFLKQDTYDVHLSLSDH
GNKEQLTVIRATVCDCHGHVETCPGPWKGG
HER2
(SEQ ID NO: 40)
TQVCTGTDMKLRLPASPETHLDMLRHLYQGCQVVQGNLELTYLPTNASLSFLQDIQEVQGYVLIAHNQVRQVPLQ
RLRIVRGTQLFEDNYALAVLDNGDPLNNTTPVTGASPGGLRELQLRSLTEILKGGVLIQRNPQLCYQDTILWKDI
FHKNNQLALTLIDTNRSRACHPCSPMCKGSRCWGESSEDCQSLTRTVCAGGCARCKGPLPTDCCHEQCAAGCTGP
KHSDCLACLHFNHSGICELHCPALVTYNTDTFESMPNPEGRYTFGASCVTACPYNYLSTDVGSCTLVCPLHNQEV
TAEDGTQRCEKCSKPCARVCYGLGMEHLREVRAVTSANIQEFAGCKKIFGSLAFLPESFDGDPASNTAPLQPEQL
QVFETLEEITGYLYISAWPDSLPDLSVFQNLQVIRGRILHNGAYSLTLQGLGISWLGLRSLRELGSGLALIHHNT
HLCFVHTVPWDQLFRNPHQALLHTANRPEDECVGEGLACHQLCARGHCWGPGPTQCVNCSQFLRGQECVEECRVL
QGLPREYVNARHCLPCHPECQPQNGSVTCFGPEADQCVACAHYKDPPFCVARCPSGVKPDLSYMPIWKEPDEEGA
CQPCPINCTHSCVDLDDKGCPAEQRASPLT
IGFBP2
(SEQ ID NO: 41)
EVLFRCPPCTPERLAACGPPPVAPPAAVAAVAGGARMPCAELVREPGCGCCSVCARLEGEACGVYTPRCGQGLRC
YPHPGSELPLQALVMGEGTCEKRRDAEYGASPEQVADNGDDHSEGGLVENHVDSTMNMLGGGGSAGRKPLKSGMK
ELAVFREKVTEQHRQMGKGGKHHLGLEEPKKLRPPPARTPCQQELDQVLERISTMRLPDERGPLEHLYSLHIPNC
DKHGLYNLKQCKMSLNGQRGECWCVNPNTGKLIQGAPTIRGDPECHLFYNEQQEARGVHTQRMQ
SPINT2
(SEQ ID NO: 42)
ADRERSIHDFCLVSKVVGRCRASMPRWWYNVTDGSCQLFVYGGCDGNSNNYLTKEECLKKCATVTENATGDLATS
RNAADSSVPSAPRRQDSEDHSSDMFNYEEYCTANAVTGPCRASFPRWYFDVERNSCNNFIYGGCRGNKNSYRSEE
ACMLRCFRQQENPPLPLGSKV
SUSD2
(SEQ ID NO: 43)
QESCSMRCGALDGPCSCHPTCSGLGTCCLDFRDFCLEILPYSGSMMGGKDFVVRHFKMSSPTDASVICRFKDSIQ
TLGHVDSSGQVHCVSPLLYESGRIPFTVSLDNGHSFPRAGTWLAVHPNKVSMMEKSELVNETRWQYYGTANTSGN
LSLTWHVKSLPTQTITIELWGYEETGMPYSQEWTAKWSYLYPLATHIPNSGSFTFTPKPAPPSYQRWRVGALRII
DSKNYAGQKDVQALWTNDHALAWHLSDDFREDPVAWARTQCQAWEELEDQLPNFLEELPDCPCTLTQARADSGRF
FTDYGCDMEQGSVCTYHPGAVHCVRSVQASLRYGSGQQCCYTADGTQLLTADSSGGSTPDRGHDWGAPPFRTPPR
VPSMSHWLYDVLSFYYCCLWAPDCPRYMQRRPSNDCRNYRPPRLASAFGDPHFVTFDGTNFTFNGRGEYVLLEAA
LTDLRVQARAQPGTMSNGTETRGTGLTAVAVQEGNSDVVEVRLANRTGGLEVLLNQEVLSFTEQSWMDLKGMFLS
VAAGDRVSIMLASGAGLEVSVQGPFLSVSVLLPEKFLTHTHGLLGTLNNDPTDDFTLHSGRVLPPGTSPQELFLF
GANWTVHNASSLLTYDSWFLVHNFLYQPKHDPTFEPLFPSETTLNPSLAQEAAKLCGDDHFCNFDVAATGSLSTG
TATRVAHQLHQRRMQSLQPVVSCGWLAPPPNGQKEGNRYLAGSTIYFHCDNGYSLAGAETSTCQADGTWSSPTPK
CQPGRSYA
CALD1 sequence
(SEQ ID NO: 44)
MLGGSGSHGRRSLAALSQIAYQRNDDDEEEAARERRRRARQERLRQKQEEESLGQVTDQVEVNAQNSVPDEEAKT
TTTNTQVEGDDEAAFLERLARREERRQKRLQEALERQKEFDPTITDASLSLPSRRMQNDTAENETTEKEEKSESR
QERYEIEETETVTKSYQKNDWRDAEENKKEDKEKEEEEEEKPKRGSIGENQGEEKGTKVQAKREKLQEDKPTFKK
EEIKDEKIKKDKEPKEEVKSFMDRKKGFTEVKSQNGEFMTHKLKHTENTFSRPGGRASVDTKEAEGAPQVEAGKR
LEELRRRRGETESEEFEKLKQKQQEAALELEELKKKREERRKVLEEEEQRRKQEEADRKLREEEEKRRLKEEIER
RRAEAAEKRQKMPEDGLSDDKKPFKCFTPKGSSLKIEERAEFLNKSVQKSSGVKSTHQAAIVSKIDSRLEQYTSA
IEGTKSAKPTKPAASDLPVPAEGVRNIKSMWEKGNVFSSPTAAGTPNKETAGLKVGVSSRINEWLTKTPDGNKSP
APKPSDLRPGDVSSKRNLWEKQSVDKVTSPTKV
XAGE1B sequence
(SEQ ID NO: 45)
MESPKKKNQQLKVGILHLGSRQKKIRIQLRSQCATWKVICKSCISQTPGINLDLGSGVKVKIIPKEEHCKMPEAG
EEQPQV
BIRC5 sequence
(SEQ ID NO: 46)
MGAPTLPPAWQPFLKDHRISTFKNWPFLEGCACTPERMAEAGFIHCPTENEPDLAQCFFCFKELEGWEPDDDPIE
EHKKHSSGCAFLSVKKQFEELTLGEFLKLDRERAKNKIAKETNNKKKEFEETAEKVRRAIEQLAAMD
LGALS3 sequence
(SEQ ID NO: 47)
MADNFSLHDALSGSGNPNPQGWPGAWGNQPAGAGGYPGASYPGAYPGQAPPGAYPGQAPPGAYPGAPGAYPGAPA
PGVYPGPPSGPGAYPSSGQPSATGAYPATGPYGAPAGPLIVPYNLPLPGGVVPRMLITILGTVKPNANRIALDFQ
RGNDVAFHFNPRFNENNRRVIVCNTKLDNNWGREERQSVFPFESGKPFKIQVLVEPDHFKVAVNDAHLLQYNHRV
KKLNEISKLGISGDIDLTSASYTMI

In various embodiments, the one or more first antibody detection markers bind to human autoantibodies against at least 2, 3, 4, 5, 6, 7, 8, 9, 10, 11, 12, 13, 14, 15, 16, 17, 18, or more ENA selected from the group consisting of CENPA, Jo-1-U1-snRNP subunit 70 kD, U1-snRNP subunit A, U1-snRNP subunit C, Sm subunit B, Sm subunit B′, Sm subunit D, Sm subunit E, Sm subunit F, Sm subunit G (referred to cumulatively herein as “Sm”), Scl70, SS-A/Ro (52 kDa, 60 kDa, or both), SS-B/La, CENPB, U1-snRNP, PM/Scl100, Ku, and PM/Scl75. In some embodiments, antibody detection marker may bind to human autoantibodies against Sm or U1 snRNP (i.e.: without distinguishing between different subunits) in other embodiments, the antibody detection marker may bind to human autoantibodies against specific or Sm or U1 snRNP subunits.

In other embodiments, the one or more second antibody detection markers bind to human autoantibodies against at least 2, 3, 4, 5, 6, 7, 8, 9, 10, 11, 12, 13, 14, 15, 16, 17, 18, 19, 20, 21, 22, 23, 24, 25, 26, or all 27 TAAs selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, and GRP78.

In one embodiment, the one or more ENA comprises 1, 2, 3, or all 4 of SS-A/Ro (52 kDa), SS-B/La, CENPB, and Ku. In another embodiment, the one or more ENA comprises 1, 2, or all 3 of SS-A/Ro, SS-B/LA, and Ku.

In one embodiment, the one or more TAA comprises 1, 2, 3, 4, 5, 6, 7, 8, 9, 10, or all 11 of ANGPTL4, CALD1, CD320, CDH3, DKK1, LRRC15, MUC1, SPINT2, SPON2, SSR2, and GRP78. In another embodiment, the one or more TAA comprises 1, 2, 3, 4, 5, 6, or all 7 of DKK1, ANGPTL4, MUC1, GRP78, GFRA1, A1AT, and GRN.

In various further embodiments, the combination of one or more TAA and one or more ENA comprises a set selected from the group consisting of:

A1AT and Sm;

A1AT and IGFBP2;

A1AT and GRN;

A1AT and CENPA;

A1AT, Sm, and IGFPB2;

A1AT, Sm, and GRN;

A1AT, Sm, and CENPA;

A1AT, Sm, IGFBP2, and GRN;

A1AT, Sm, IGFBP2, and CENPA

A1AT, Sm, IGFBP2, GRN, and CENPA;

A1AT, IGFBP2, and GRN;

A1AT, IGFBP2, and CENPA;

A1AT, IGFBP2, GRN, and CENPA;

Sm and IGFPB2;

Sm and GRN;

Sm and CENPA;

Sm, IGFPB2, and GRN;

Sm, IGFPB2, and CENPA;

Sm, IGFPB2, GRN, and CENPA;

IGFPB2 and GRN;

IGFPB2 and CENPA;

IGFPB2, GRN, and CENPA;

GRN and CENPA;

DKK1 and SS-A/Ro;

DKKI and SS-B/La;

DKKI and Ku;

DKKI, SS-A/Ro, and SS-B/La;

DKK1, SS-A/Ro, and Ku;

DKK1, SS-B/La, and Ku;

DKK1, ANGPTL4, and SS-A/Ro;

DKKI, ANGPTL4, and SS-B/La;

DKKI, ANGPTL4, and Ku;

DKKI, ANGPTL4, SS-A/Ro, and SS-B/La;

DKK1, ANGPTL4, SS-A/Ro, and Ku;

DKK1, ANGPTL4, SS-B/La, and Ku;

DKK1, MUC1, and SS-A/Ro;

DKKI, MUC1, and SS-B/La;

DKKI, MUC1, and Ku;

DKKI, MUC1, SS-A/Ro, and SS-B/La;

DKK1, MUC1, SS-A/Ro, and Ku;

DKK1, MUC1, SS-B/La, and Ku;

DKK1, ANGPTL4, MUC1, and SS-A/Ro;

DKKI, ANGPTL4, MUC1, and SS-B/La;

DKKI, ANGPTL4, MUC1, and Ku;

DKKI, ANGPTL4, MUC1, SS-A/Ro, and SS-B/La;

DKK1, ANGPTL4, MUC1, SS-A/Ro, and Ku;

DKK1, ANGPTL4, MUC1, SS-B/La, and Ku;

DKK1, GRP78, and SS-A/Ro;

DKKI, GRP78, and SS-B/La;

DKKI, GRP78, and Ku;

DKKI, GRP78, SS-A/Ro, and SS-B/La;

DKK1, GRP78, SS-A/Ro, and Ku;

DKK1, GRP78, SS-B/La, and Ku;

DKK1, ANGPTL4, GRP78, and SS-A/Ro;

DKKI, ANGPTL4, GRP78, and SS-B/La;

DKKI, ANGPTL4, GRP78, and Ku;

DKKI, ANGPTL4, GRP78, SS-A/Ro, and SS-B/La;

DKK1, ANGPTL4, GRP78, SS-A/Ro, and Ku;

DKK1, ANGPTL4, GRP78, SS-B/La, and Ku;

DKK1, MUC1, GRP78, and SS-A/Ro;

DKKI, MUC1, GRP78, and SS-B/La;

DKKI, MUC1, GRP78, and Ku;

DKKI, MUC1, GRP78, SS-A/Ro, and SS-B/La;

DKK1, MUC1, GRP78, SS-A/Ro, and Ku;

DKK1, MUC1, GRP78, SS-B/La, and Ku;

DKK1, ANGPTL4, MUC1, GRP78, and SS-A/Ro;

DKKI, ANGPTL4, MUC1, GRP78, and SS-B/La;

DKKI, ANGPTL4, MUC1, GRP78, and Ku;

DKKI, ANGPTL4, MUC1, GRP78, SS-A/Ro, and SS-B/La;

DKK1, ANGPTL4, MUC1, GRP78, SS-A/Ro, and Ku;

DKK1, ANGPTL4, MUC1, GRP78, SS-B/La, and Ku;

DKK1, GFRA1, and SS-A/Ro;

DKKI, GFRA1, and SS-B/La;

DKKI, GFRA1, and Ku;

DKKI, GFRA1, SS-A/Ro, and SS-B/La;

DKK1, GFRA1, SS-A/Ro, and Ku;

DKK1, GFRA1, SS-B/La, and Ku;

DKK1, ANGPTL4, GFRA1, and SS-A/Ro;

DKKI, ANGPTL4, GFRA1, and SS-B/La;

DKKI, ANGPTL4, GFRA1, and Ku;

DKKI, ANGPTL4, GFRA1, SS-A/Ro, and SS-B/La;

DKK1, ANGPTL4, GFRA1, SS-A/Ro, and Ku;

DKK1, ANGPTL4, GFRA1, SS-B/La, and Ku;

DKK1, MUC1, GFRA1, and SS-A/Ro;

DKKI, MUC1, GFRA1, and SS-B/La;

DKKI, MUC1, GFRA1, and Ku;

DKKI, MUC1, GFRA1, SS-A/Ro, and SS-B/La;

DKK1, MUC1, GFRA1, SS-A/Ro, and Ku;

DKK1, MUC1, GFRA1, SS-B/La, and Ku;

DKK1, ANGPTL4, MUC1, GFRA1, and SS-A/Ro;

DKKI, ANGPTL4, MUC1, GFRA1, and SS-B/La;

DKKI, ANGPTL4, MUC1, GFRA1, and Ku;

DKKI, ANGPTL4, MUC1, GFRA1, SS-A/Ro, and SS-B/La;

DKK1, ANGPTL4, MUC1, GFRA1, SS-A/Ro, and Ku;

DKK1, ANGPTL4, MUC1, GFRA1, SS-B/La, and Ku;

DKK1, ANGPTL4, MUC1, GRP78, GFRA1, and SS-A/Ro;

DKKI, ANGPTL4, MUC1, GRP78, GFRA1, and SS-B/La;

DKKI, ANGPTL4, MUC1, GRP78, GFRA1, and Ku;

DKKI, ANGPTL4, MUC1, GRP78, GFRA1, SS-A/Ro, and SS-B/La;

DKK1, ANGPTL4, MUC1, GRP78, GFRA1, SS-A/Ro, and Ku;

DKK1, ANGPTL4, MUC1, GRP78, GFRA1, SS-B/La, and Ku;

DKK1, A1AT, and SS-A/Ro;

DKKI, A1AT, and SS-B/La;

DKKI, A1AT, and Ku;

DKKI, A1AT, SS-A/Ro, and SS-B/La;

DKK1, A1AT, SS-A/Ro, and Ku;

DKK1, A1AT, SS-B/La, and Ku;

DKK1, ANGPTL4, A1AT, and SS-A/Ro;

DKKI, ANGPTL4, A1AT, and SS-B/La;

DKKI, ANGPTL4, A1AT, and Ku;

DKKI, ANGPTL4, A1AT, SS-A/Ro, and SS-B/La;

DKK1, ANGPTL4, A1AT, SS-A/Ro, and Ku;

DKK1, ANGPTL4, A1AT, SS-B/La, and Ku;

DKK1, MUC1, A1AT, and SS-A/Ro;

DKKI, MUC1, A1AT, and SS-B/La;

DKKI, MUC1, A1AT, and Ku;

DKKI, MUC1, A1AT, SS-A/Ro, and SS-B/La;

DKK1, MUC1, A1AT, SS-A/Ro, and Ku;

DKK1, MUC1, A1AT, SS-B/La, and Ku;

DKK1, ANGPTL4, MUC1, A1AT, and SS-A/Ro;

DKKI, ANGPTL4, MUC1, A1AT, and SS-B/La;

DKKI, ANGPTL4, MUC1, A1AT, and Ku;

DKKI, ANGPTL4, MUC1, A1AT, SS-A/Ro, and SS-B/La;

DKK1, ANGPTL4, MUC1, A1AT, SS-A/Ro, and Ku;

DKK1, ANGPTL4, MUC1, A1AT, SS-B/La, and Ku;

DKK1, ANGPTL4, MUC1, GRP78, A1AT, and SS-A/Ro;

DKKI, ANGPTL4, MUC1, GRP78, A1AT, and SS-B/La;

DKKI, ANGPTL4, MUC1, GRP78, A1AT, and Ku;

DKKI, ANGPTL4, MUC1, GRP78, A1AT, SS-A/Ro, and SS-B/La;

DKK1, ANGPTL4, MUC1, GRP78, A1AT, SS-A/Ro, and Ku;

DKK1, ANGPTL4, MUC1, GRP78, A1AT, SS-B/La, and Ku;

DKK1, ANGPTL4, MUC1, GRP78, GFRA1, A1AT, and SS-A/Ro;

DKKI, ANGPTL4, MUC1, GRP78, GFRA1, A1AT, and SS-B/La;

DKKI, ANGPTL4, MUC1, GRP78, GFRA1, A1AT, and Ku;

DKKI, ANGPTL4, MUC1, GRP78, GFRA1, A1AT, SS-A/Ro, and SS-B/La;

DKK1, ANGPTL4, MUC1, GRP78, GFRA1, A1AT, SS-A/Ro, and Ku;

DKK1, ANGPTL4, MUC1, GRP78, GFRA1, A1AT, SS-B/La, and Ku;

DKK1, GRN, and SS-A/Ro;

DKKI, GRN, and SS-B/La;

DKKI, GRN, and Ku;

DKKI, GRN, SS-A/Ro, and SS-B/La;

DKK1, GRN, SS-A/Ro, and Ku;

DKK1, GRN, SS-B/La, and Ku;

DKK1, ANGPTL4, GRN, and SS-A/Ro;

DKKI, ANGPTL4, GRN, and SS-B/La;

DKKI, ANGPTL4, GRN, and Ku;

DKKI, ANGPTL4, GRN, SS-A/Ro, and SS-B/La;

DKK1, ANGPTL4, GRN, SS-A/Ro, and Ku;

DKK1, ANGPTL4, GRN, SS-B/La, and Ku;

DKK1, MUC1, GRN, and SS-A/Ro;

DKKI, MUC1, GRN, and SS-B/La;

DKKI, MUC1, GRN, and Ku;

DKKI, MUC1, GRN, SS-A/Ro, and SS-B/La;

DKK1, MUC1, GRN, SS-A/Ro, and Ku;

DKK1, MUC1, GRN, SS-B/La, and Ku;

DKK1, ANGPTL4, MUC1, GRN, and SS-A/Ro;

DKKI, ANGPTL4, MUC1, GRN, and SS-B/La;

DKKI, ANGPTL4, MUC1, GRN, and Ku;

DKKI, ANGPTL4, MUC1, GRN, SS-A/Ro, and SS-B/La;

DKK1, ANGPTL4, MUC1, GRN, SS-A/Ro, and Ku;

DKK1, ANGPTL4, MUC1, GRN, SS-B/La, and Ku;

DKK1, ANGPTL4, MUC1, GRP78, GRN, and SS-A/Ro;

DKKI, ANGPTL4, MUC1, GRP78, GRN, and SS-B/La;

DKKI, ANGPTL4, MUC1, GRP78, GRN, and Ku;

DKKI, ANGPTL4, MUC1, GRP78, GRN, SS-A/Ro, and SS-B/La;

DKK1, ANGPTL4, MUC1, GRP78, GRN, SS-A/Ro, and Ku;

DKK1, ANGPTL4, MUC1, GRP78, GRN, SS-B/La, and Ku;

DKK1, ANGPTL4, MUC1, GRP78, GFRA1, GRN, and SS-A/Ro;

DKKI, ANGPTL4, MUC1, GRP78, GFRA1, GRN, and SS-B/La;

DKKI, ANGPTL4, MUC1, GRP78, GFRA1, GRN, and Ku;

DKKI, ANGPTL4, MUC1, GRP78, GFRA1, GRN, SS-A/Ro, and SS-B/La;

DKK1, ANGPTL4, MUC1, GRP78, GFRA1, GRN, SS-A/Ro, and Ku;

DKK1, ANGPTL4, MUC1, GRP78, GFRA1, GRN, SS-B/La, and Ku;

DKK1, ANGPTL4, MUC1, GRP78, GFRA1, A1AT, GRN, and SS-A/Ro;

DKKI, ANGPTL4, MUC1, GRP78, GFRA1, A1AT, GRN, and SS-B/La;

DKKI, ANGPTL4, MUC1, GRP78, GFRA1, A1AT, GRN, and Ku;

DKKI, ANGPTL4, MUC1, GRP78, GFRA1, A1AT, GRN, SS-A/Ro, and SS-B/La;

DKK1, ANGPTL4, MUC1, GRP78, GFRA1, A1AT, GRN, SS-A/Ro, and Ku;

DKK1, ANGPTL4, MUC1, GRP78, GFRA1, A1AT, GRN, SS-B/La, and Ku;

ANGPTL4 and SS-A/Ro;

ANGPTL4 and SS-B/La;

ANGPTL4 and Ku;

ANGPTL4, SS-A/Ro, and SS-B/La;

ANGPTL4, SS-A/Ro, and Ku;

ANGPTL4, SS-B/La, and Ku;

ANGPTL4, MUC1, and SS-A/Ro;

ANGPTL4, MUC1, and SS-B/La;

ANGPTL4, MUC1, and Ku;

ANGPTL4, MUC1, SS-A/Ro, and SS-B/La;

ANGPTL4, MUC1, SS-A/Ro, and Ku;

ANGPTL4, MUC1, SS-B/La, and Ku;

ANGPTL4, GRP78, and SS-A/Ro;

ANGPTL4, GRP78, and SS-B/La;

ANGPTL4, GRP78, and Ku;

ANGPTL4, GRP78, SS-A/Ro, and SS-B/La;

ANGPTL4, GRP78, SS-A/Ro, and Ku;

ANGPTL4, GRP78, SS-B/La, and Ku;

ANGPTL4, MUC1, GRP78, and SS-A/Ro;

ANGPTL4, MUC1, GRP78, and SS-B/La;

ANGPTL4, MUC1, GRP78, and Ku;

ANGPTL4, MUC1, GRP78, SS-A/Ro, and SS-B/La;

ANGPTL4, MUC1, GRP78, SS-A/Ro, and Ku;

ANGPTL4, MUC1, GRP78, SS-B/La, and Ku;

ANGPTL4, GFRA1, and SS-A/Ro;

ANGPTL4, GFRA1, and SS-B/La;

ANGPTL4, GFRA1, and Ku;

ANGPTL4, GFRA1, SS-A/Ro, and SS-B/La;

ANGPTL4, GFRA1, SS-A/Ro, and Ku;

ANGPTL4, GFRA1, SS-B/La, and Ku;

ANGPTL4, MUC1, GFRA1, and SS-A/Ro;

ANGPTL4, MUC1, GFRA1, and SS-B/La;

ANGPTL4, MUC1, GFRA1, and Ku;

ANGPTL4, MUC1, GFRA1, SS-A/Ro, and SS-B/La;

ANGPTL4, MUC1, GFRA1, SS-A/Ro, and Ku;

ANGPTL4, MUC1, GFRA1, SS-B/La, and Ku;

ANGPTL4, MUC1, GRP78, GFRA1, and SS-A/Ro;

ANGPTL4, MUC1, GRP78, GFRA1, and SS-B/La;

ANGPTL4, MUC1, GRP78, GFRA1, and Ku;

ANGPTL4, MUC1, GRP78, GFRA1, SS-A/Ro, and SS-B/La;

ANGPTL4, MUC1, GRP78, GFRA1, SS-A/Ro, and Ku;

ANGPTL4, MUC1, GRP78, GFRA1, SS-B/La, and Ku;

ANGPTL4, A1AT, and SS-A/Ro;

ANGPTL4, A1AT, and SS-B/La;

ANGPTL4, A1AT, and Ku;

ANGPTL4, A1AT, SS-A/Ro, and SS-B/La;

ANGPTL4, A1AT, SS-A/Ro, and Ku;

ANGPTL4, A1AT, SS-B/La, and Ku;

ANGPTL4, MUC1, A1AT, and SS-A/Ro;

ANGPTL4, MUC1, A1AT, and SS-B/La;

ANGPTL4, MUC1, A1AT, and Ku;

ANGPTL4, MUC1, A1AT, SS-A/Ro, and SS-B/La;

ANGPTL4, MUC1, A1AT, SS-A/Ro, and Ku;

ANGPTL4, MUC1, A1AT, SS-B/La, and Ku;

ANGPTL4, MUC1, GRP78, A1AT, and SS-A/Ro;

ANGPTL4, MUC1, GRP78, A1AT, and SS-B/La;

ANGPTL4, MUC1, GRP78, A1AT, and Ku;

ANGPTL4, MUC1, GRP78, A1AT, SS-A/Ro, and SS-B/La;

ANGPTL4, MUC1, GRP78, A1AT, SS-A/Ro, and Ku;

ANGPTL4, MUC1, GRP78, A1AT, SS-B/La, and Ku;

ANGPTL4, MUC1, GRP78, GFRA1, A1AT, and SS-A/Ro;

ANGPTL4, MUC1, GRP78, GFRA1, A1AT, and SS-B/La;

ANGPTL4, MUC1, GRP78, GFRA1, A1AT, and Ku;

ANGPTL4, MUC1, GRP78, GFRA1, A1AT, SS-A/Ro, and SS-B/La;

ANGPTL4, MUC1, GRP78, GFRA1, A1AT, SS-A/Ro, and Ku;

ANGPTL4, MUC1, GRP78, GFRA1, A1AT, SS-B/La, and Ku;

ANGPTL4, GRN, and SS-A/Ro;

ANGPTL4, GRN, and SS-B/La;

ANGPTL4, GRN, and Ku;

ANGPTL4, GRN, SS-A/Ro, and SS-B/La;

ANGPTL4, GRN, SS-A/Ro, and Ku;

ANGPTL4, GRN, SS-B/La, and Ku;

ANGPTL4, MUC1, GRN, and SS-A/Ro;

ANGPTL4, MUC1, GRN, and SS-B/La;

ANGPTL4, MUC1, GRN, and Ku;

ANGPTL4, MUC1, GRN, SS-A/Ro, and SS-B/La;

ANGPTL4, MUC1, GRN, SS-A/Ro, and Ku;

ANGPTL4, MUC1, GRN, SS-B/La, and Ku;

ANGPTL4, MUC1, GRP78, GRN, and SS-A/Ro;

ANGPTL4, MUC1, GRP78, GRN, and SS-B/La;

ANGPTL4, MUC1, GRP78, GRN, and Ku;

ANGPTL4, MUC1, GRP78, GRN, SS-A/Ro, and SS-B/La;

ANGPTL4, MUC1, GRP78, GRN, SS-A/Ro, and Ku;

ANGPTL4, MUC1, GRP78, GRN, SS-B/La, and Ku;

ANGPTL4, MUC1, GRP78, GFRA1, GRN, and SS-A/Ro;

ANGPTL4, MUC1, GRP78, GFRA1, GRN, and SS-B/La;

ANGPTL4, MUC1, GRP78, GFRA1, GRN, and Ku;

ANGPTL4, MUC1, GRP78, GFRA1, GRN, SS-A/Ro, and SS-B/La;

ANGPTL4, MUC1, GRP78, GFRA1, GRN, SS-A/Ro, and Ku;

ANGPTL4, MUC1, GRP78, GFRA1, GRN, SS-B/La, and Ku;

ANGPTL4, MUC1, GRP78, GFRA1, A1AT, GRN, and SS-A/Ro;

ANGPTL4, MUC1, GRP78, GFRA1, A1AT, GRN, and SS-B/La;

ANGPTL4, MUC1, GRP78, GFRA1, A1AT, GRN, and Ku;

ANGPTL4, MUC1, GRP78, GFRA1, A1AT, GRN, SS-A/Ro, and SS-B/La;

ANGPTL4, MUC1, GRP78, GFRA1, A1AT, GRN, SS-A/Ro, and Ku;

ANGPTL4, MUC1, GRP78, GFRA1, A1AT, GRN, SS-B/La, and Ku;

MUC1 and SS-A/Ro;

MUC1 and SS-B/La;

MUC1 and Ku;

MUC1, SS-A/Ro, and SS-B/La;

MUC1, SS-A/Ro, and Ku;

MUC1, SS-B/La, and Ku;

MUC1, GRP78, and SS-A/Ro;

MUC1, GRP78, and SS-B/La;

MUC1, GRP78, and Ku;

MUC1, GRP78, SS-A/Ro, and SS-B/La;

MUC1, GRP78, SS-A/Ro, and Ku;

MUC1, GRP78, SS-B/La, and Ku;

MUC1, GFRA1, and SS-A/Ro;

MUC1, GFRA1, and SS-B/La;

MUC1, GFRA1, and Ku;

MUC1, GFRA1, SS-A/Ro, and SS-B/La;

MUC1, GFRA1, SS-A/Ro, and Ku;

MUC1, GFRA1, SS-B/La, and Ku;

MUC1, GRP78, GFRA1, and SS-A/Ro;

MUC1, GRP78, GFRA1, and SS-B/La;

MUC1, GRP78, GFRA1, and Ku;

MUC1, GRP78, GFRA1, SS-A/Ro, and SS-B/La;

MUC1, GRP78, GFRA1, SS-A/Ro, and Ku;

MUC1, GRP78, GFRA1, SS-B/La, and Ku;

MUC1, A1AT, and SS-A/Ro;

MUC1, A1AT, and SS-B/La;

MUC1, A1 AT, and Ku;

MUC1, A1AT, SS-A/Ro, and SS-B/La;

MUC1, A1AT, SS-A/Ro, and Ku;

MUC1, A1AT, SS-B/La, and Ku;

MUC1, GRP78, A1AT, and SS-A/Ro;

MUC1, GRP78, A1AT, and SS-B/La;

MUC1, GRP78, A1AT, and Ku;

MUC1, GRP78, A1AT, SS-A/Ro, and SS-B/La;

MUC1, GRP78, A1AT, SS-A/Ro, and Ku;

MUC1, GRP78, A1AT, SS-B/La, and Ku;

MUC1, GRP78, GFRA1, A1AT, and SS-A/Ro;

MUC1, GRP78, GFRA1, A1AT, and SS-B/La;

MUC1, GRP78, GFRA1, A1AT, and Ku;

MUC1, GRP78, GFRA1, A1AT, SS-A/Ro, and SS-B/La;

MUC1, GRP78, GFRA1, A1AT, SS-A/Ro, and Ku;

MUC1, GRP78, GFRA1, A1AT, SS-B/La, and Ku;

MUC1, GRN, and SS-A/Ro;

MUC1, GRN, and SS-B/La;

MUC1, GRN, and Ku;

MUC1, GRN, SS-A/Ro, and SS-B/La;

MUC1, GRN, SS-A/Ro, and Ku;

MUC1, GRN, SS-B/La, and Ku;

MUC1, GRP78, GRN, and SS-A/Ro;

MUC1, GRP78, GRN, and SS-B/La;

MUC1, GRP78, GRN, and Ku;

MUC1, GRP78, GRN, SS-A/Ro, and SS-B/La;

MUC1, GRP78, GRN, SS-A/Ro, and Ku;

MUC1, GRP78, GRN, SS-B/La, and Ku;

MUC1, GRP78, GFRA1, GRN, and SS-A/Ro;

MUC1, GRP78, GFRA1, GRN, and SS-B/La;

MUC1, GRP78, GFRA1, GRN, and Ku;

MUC1, GRP78, GFRA1, GRN, SS-A/Ro, and SS-B/La;

MUC1, GRP78, GFRA1, GRN, SS-A/Ro, and Ku;

MUC1, GRP78, GFRA1, GRN, SS-B/La, and Ku;

MUC1, GRP78, GFRA1, A1AT, GRN, and SS-A/Ro;

MUC1, GRP78, GFRA1, A1AT, GRN, and SS-B/La;

MUC1, GRP78, GFRA1, A1AT, GRN, and Ku;

MUC1, GRP78, GFRA1, A1AT, GRN, SS-A/Ro, and SS-B/La;

MUC1, GRP78, GFRA1, A1AT, GRN, SS-A/Ro, and Ku;

MUC1, GRP78, GFRA1, A1AT, GRN, SS-B/La, and Ku;

GRP78 and SS-A/Ro;

GRP78 and SS-B/La;

GRP78 and Ku;

GRP78, SS-A/Ro, and SS-B/La;

GRP78, SS-A/Ro, and Ku;

GRP78, SS-B/La, and Ku;

GRP78, GFRA1, and SS-A/Ro;

GRP78, GFRA1, and SS-B/La;

GRP78, GFRA1, and Ku;

GRP78, GFRA1, SS-A/Ro, and SS-B/La;

GRP78, GFRA1, SS-A/Ro, and Ku;

GRP78, GFRA1, SS-B/La, and Ku;

GRP78, A1AT, and SS-A/Ro;

GRP78, A1AT, and SS-B/La;

GRP78, A1AT, and Ku;

GRP78, A1AT, SS-A/Ro, and SS-B/La;

GRP78, A1AT, SS-A/Ro, and Ku;

GRP78, A1AT, SS-B/La, and Ku;

GRP78, GFRA1, A1AT, and SS-A/Ro;

GRP78, GFRA1, A1AT, and SS-B/La;

GRP78, GFRA1, A1AT, and Ku;

GRP78, GFRA1, A1AT, SS-A/Ro, and SS-B/La;

GRP78, GFRA1, A1AT, SS-A/Ro, and Ku;

GRP78, GFRA1, A1AT, SS-B/La, and Ku;

GRP78, GRN, and SS-A/Ro;

GRP78, GRN, and SS-B/La;

GRP78, GRN, and Ku;

GRP78, GRN, SS-A/Ro, and SS-B/La;

GRP78, GRN, SS-A/Ro, and Ku;

GRP78, GRN, SS-B/La, and Ku;

GRP78, GFRA1, GRN, and SS-A/Ro;

GRP78, GFRA1, GRN, and SS-B/La;

GRP78, GFRA1, GRN, and Ku;

GRP78, GFRA1, GRN, SS-A/Ro, and SS-B/La;

GRP78, GFRA1, GRN, SS-A/Ro, and Ku;

GRP78, GFRA1, GRN, SS-B/La, and Ku;

GRP78, GFRA1, A1AT, GRN, and SS-A/Ro;

GRP78, GFRA1, A1AT, GRN, and SS-B/La;

GRP78, GFRA1, A1AT, GRN, and Ku;

GRP78, GFRA1, A1AT, GRN, SS-A/Ro, and SS-B/La;

GRP78, GFRA1, A1AT, GRN, SS-A/Ro, and Ku;

GRP78, GFRA1, A1AT, GRN, SS-B/La, and Ku;

GFRA1 and SS-A/Ro;

GFRA1 and SS-B/La;

GFRA1 and Ku;

GFRA1, SS-A/Ro, and SS-B/La;

GFRA1, SS-A/Ro, and Ku;

GFRA1, SS-B/La, and Ku;

GFRA1, A1AT, and SS-A/Ro;

GFRA1, A1AT, and SS-B/La;

GFRA1, A1AT, and Ku;

GFRA1, A1AT, SS-A/Ro, and SS-B/La;

GFRA1, A1AT, SS-A/Ro, and Ku;

GFRA1, A1AT, SS-B/La, and Ku;

GFRA1, GRN, and SS-A/Ro;

GFRA1, GRN, and SS-B/La;

GFRA1, GRN, and Ku;

GFRA1, GRN, SS-A/Ro, and SS-B/La;

GFRA1, GRN, SS-A/Ro, and Ku;

GFRA1, GRN, SS-B/La, and Ku;

GFRA1, A1AT, GRN, and SS-A/Ro;

GFRA1, A1AT, GRN, and SS-B/La;

GFRA1, A1AT, GRN, and Ku;

GFRA1, A1AT, GRN, SS-A/Ro, and SS-B/La;

GFRA1, A1AT, GRN, SS-A/Ro, and Ku;

GFRA1, A1AT, GRN, SS-B/La, and Ku;

A1AT and SS-A/Ro;

A1AT and SS-B/La;

A1AT and Ku;

A1AT, SS-A/Ro, and SS-B/La;

A1AT, SS-A/Ro, and Ku;

A1AT, SS-B/La, and Ku;

A1AT, GRN, and SS-A/Ro;

A1AT, GRN, and SS-B/La;

A1AT, GRN, and Ku;

A1AT, GRN, SS-A/Ro, and SS-B/La;

A1AT, GRN, SS-A/Ro, and Ku; and

A1AT, GRN, SS-B/La, and Ku.

The methods may be used to diagnose cancer, prognose cancer occurrence or recurrence, and/or monitor cancer therapy. In one embodiment, the method may be used to diagnose cancer, and the subject is a subject at risk of having cancer. In a further embodiment the subject is at risk of having breast cancer. In this embodiment, the subject may be any subject at risk of having breast cancer, including but not limited to subjects (such as human subjects) with a family history of breast cancer or other genetic predisposition to cancer, or has one or more symptoms of breast cancer.

In another embodiment, the subject (such as a human subject) was previously treated for cancer, such as breast cancer, and is thus at risk of breast cancer recurrence.

In a further embodiment, the subject (such as a human subject) is being treated for cancer, such as breast cancer, and the methods may be used to monitor efficacy of the treatment, including but not limited to chemotherapy, hormonal therapy, radiation therapy, antibody therapies or other targeted therapies, and/or surgery to remove the tumor.

In another embodiment, the subject (such as a human subject) was diagnosed as having cancer (such as breast cancer) by other techniques, including but not limited to mammography, ultrasound, biopsy, MRI or other imaging modalities, wherein the method is used to identify false positive cancer diagnoses.

In all of these embodiments, the methods of the disclosure may be carried out once, or may be carried out at intervals as deemed appropriate by attending medical personnel. By way of non-limiting example, the method may be carried out a first time to establish a baseline of human autoantibodies against the one or more ENAs and TAAs, with subsequent iterations of the methods carried out to assess changes in the level of autoantibodies, as a way to monitor efficacy of therapy.

In all of these embodiments, the methods of the disclosure may combined with one or more other tests, including but not limited to mammography, physical exam, CT-Scan, PET-Scan, detection of circulating DNA, or other imaging or blood tests, to improve the accuracy and/or reliability and/or Sensitivity and/or Specificity of detecting and/or diagnosing and/or prognosing and/or monitor cancer occurrence or recurrence.

In all embodiment and combinations of embodiments herein, the antibody detection markers may be any suitable reagents that can be used to detect antibodies against the recited proteins, including but not limited to the recited protein, a secreted version of the protein (such as a native secreted form of the protein), or an extracellular domain of the protein. Secreted proteins are more easily delivered from tumor cells to lymph nodes, where interactions of immune cells take place resulting in abundant high-affinity antibodies. Membrane surface proteins are commonly released in a soluble form from tumor cells through metalloproteinase-dependent cleavage. The shed proteins are more easily transferred to the lymph nodes than intracellular protein. Thus, in one embodiment the antibody detection marker is a secreted or membrane portion of the recited protein.

In all of the above embodiments, the antibody detection markers may be labeled with a detectable label. In one embodiment, the detectable labels for reagents to detect autoantibodies against one ENA or TAA are distinguishable from the detectable labels to detect autoantibodies against other ENAs or TAAs. Methods for detecting the label include, but are not limited to spectroscopic, photochemical, biochemical, immunochemical, physical or chemical techniques. Any suitable detectable label can be used.

In one embodiment, the antibody detection markers are immobilized on a surface, including but not limited to a bead, a nanoparticle, a microarray, glass slide, membrane, microplate, etc.

In another embodiment, the antibody detection marker molecules comprise the composition as descried herein.

In all embodiments and combinations of embodiments herein, the contacting can be carried out under any suitable conditions for promoting binding between the autoantibodies in the bodily fluid sample and the antibody detection marker molecules to form a binding complex that can be detected. Appropriate such conditions can be determined by those of skill in the art based on the intended assay, in light of the teachings herein. Similarly, any suitable additional steps can be used in the methods, such as one or more wash or other steps to remove unbound reagents.

Any suitable detection technique can be used, including but not limited to enzyme linked immunosorbent assays (ELISA), bead based assay platforms such as the Luminex systems, 2-D array based assay platforms such as SearchLight®, and the Inanovate® ‘Longitudinal Assay Screening’ platform which may be capable of quantitating human autoantibodies against all the listed ENAs and TAAs from patient samples at their clinically relevant concentrations in a single test and dilution. In one embodiment, the compositions can be placed on a solid support, such as in a microarray, glass slide, membrane, microplate format or beads. The embodiment facilitates use of the compositions. Exemplary such assays are provided in the examples.

Similarly, any suitable bodily fluid can be used, including but not limited to a serum sample, plasma sample or blood sample from the subject.

In one embodiment, the method identifies the subject as likely to have cancer or cancer recurrence, and the method further comprises treating the subject with an amount of a therapeutic sufficient to treat the cancer or cancer recurrence. In one such embodiment, the treatment comprises one or more of chemotherapy, hormonal therapy, radiation therapy, and/or surgery to remove the tumor.

In another aspect, the disclosure provides antibody detection marker composition comprising

(a) one or more first antibody detection marker molecules that bind to human autoantibodies against at least one tumor associated antigen (TAA); and

(b) one or more second antibody detection marker molecules that bind to human autoantibodies against at least one extractable nuclear antigen (ENA).

All embodiments of the first antibody detection marker molecules, the second antibody detection marker molecules, the ENAs, and the TAAs disclosed in the first aspect are suitable for use in this aspect of the disclosure.

Thus, in one embodiment, the at least one ENA is selected from the group consisting of CENPA, Jo-1-U1-snRNP subunit 70 kD, U1-snRNP subunit A, U1-snRNP subunit C, Sm subunit B, Sm subunit B′, Sm subunit D, Sm subunit E, Sm subunit F, Sm subunit G (referred to cumulatively herein as “Sm”), Scl70, Sm, SS-A/Ro (52 kDa, 60 kDa, or both), SS-B/La, CENPB, U1-snRNP, PM/Scl100, Ku, and PM/Scl75. In another embodiment, the TAA may be associated with a tumor type including but not limited to breast cancer, prostate cancer, ovarian cancer, lung or any other epithelial cancer. In one specific embodiment, the at least one TAA comprises at least one breast cancer TAA.

In another specific embodiment, the at least one TAA is selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, GRN, and GRP78.

In various embodiments, the one or more first antibody detection markers bind to human autoantibodies against at least 2, 3, 4, 5, 6, 7, 8, 9, 10, 11, 12, 13, 14, 15, 16, 17, 18, or more ENA selected from the group consisting of CENPA, Jo-1, U1-snRNP subunit 70 kD, U1-snRNP subunit A, U1-snRNP subunit C, Sm subunit B, Sm subunit B′, Sm subunit D, Sm subunit E, Sm subunit F, Sm subunit G (referred to cumulatively herein as “Sm”), Scl70, SS-A/Ro (52 kDa, 60 kDa, or both), SS-B/La, CENPB, U1-snRNP, PM/Scl100, Ku, and PM/Scl75. In some embodiments, antibody detection marker may bind to human autoantibodies against Sm or U1 snRNP (i.e.: without distinguishing between different subunits) in other embodiments, the antibody detection marker may bind to human autoantibodies against specific or Sm or U1 snRNP subunits.

In other embodiments, the one or more second antibody detection markers bind to human autoantibodies against at least 2, 3, 4, 5, 6, 7, 8, 9, 10, 11, 12, 13, 14, 15, 16, 17, 18, 19, 20, 21, 22, 23, 24, 25, 26, or all 27 TAAs selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, and GRP78.

In one embodiment, the one or more ENA comprises 1, 2, 3, or all 4 of SS-A/Ro (52 kDa), SS-B/La, CENPB, and Ku. In another embodiment, the one or more ENA comprises 1, 2, or all 3 of SS-A/Ro, SS-B/LA, and Ku.

In one embodiment, the one or more TAA comprises 1, 2, 3, 4, 5, 6, 7, 8, 9, 10, or all 11 of ANGPTL4, CALD1, CD320, CDH3, DKK1, LRRC15, MUC1, SPINT2, SPON2, SSR2, and GRP78. In another embodiment, the one or more TAA comprises 1, 2, 3, 4, 5, 6, or all 7 of DKK1, ANGPTL4, MUC1, GRP78, GFRA1, A1AT, and GRN.

In various further embodiments, the combination of one or more TAA and one or more ENA comprises a set selected from the group consisting of:

A1AT and Sm;

A1AT and IGFBP2;

A1AT and GRN;

A1AT and CENPA;

A1AT, Sm, and IGFPB2;

A1AT, Sm, and GRN;

A1AT, Sm, and CENPA;

A1AT, Sm, IGFBP2, and GRN;

A1AT, Sm, IGFBP2, and CENPA

A1AT, Sm, IGFBP2, GRN, and CENPA;

A1AT, IGFBP2, and GRN;

A1AT, IGFBP2, and CENPA;

A1AT, IGFBP2, GRN, and CENPA;

Sm and IGFPB2;

Sm and GRN;

Sm and CENPA;

Sm, IGFPB2, and GRN;

Sm, IGFPB2, and CENPA;

Sm, IGFPB2, GRN, and CENPA;

IGFPB2 and GRN;

IGFPB2 and CENPA;

IGFPB2, GRN, and CENPA;

GRN and CENPA;

DKK1 and SS-A/Ro;

DKKI and SS-B/La;

DKKI and Ku;

DKKI, SS-A/Ro, and SS-B/La;

DKK1, SS-A/Ro, and Ku;

DKK1, SS-B/La, and Ku;

DKK1, ANGPTL4, and SS-A/Ro;

DKKI, ANGPTL4, and SS-B/La;

DKKI, ANGPTL4, and Ku;

DKKI, ANGPTL4, SS-A/Ro, and SS-B/La;

DKK1, ANGPTL4, SS-A/Ro, and Ku;

DKK1, ANGPTL4, SS-B/La, and Ku;

DKK1, MUC1, and SS-A/Ro;

DKKI, MUC1, and SS-B/La;

DKKI, MUC1, and Ku;

DKKI, MUC1, SS-A/Ro, and SS-B/La;

DKK1, MUC1, SS-A/Ro, and Ku;

DKK1, MUC1, SS-B/La, and Ku;

DKK1, ANGPTL4, MUC1, and SS-A/Ro;

DKKI, ANGPTL4, MUC1, and SS-B/La;

DKKI, ANGPTL4, MUC1, and Ku;

DKKI, ANGPTL4, MUC1, SS-A/Ro, and SS-B/La;

DKK1, ANGPTL4, MUC1, SS-A/Ro, and Ku;

DKK1, ANGPTL4, MUC1, SS-B/La, and Ku;

DKK1, GRP78, and SS-A/Ro;

DKKI, GRP78, and SS-B/La;

DKKI, GRP78, and Ku;

DKKI, GRP78, SS-A/Ro, and SS-B/La;

DKK1, GRP78, SS-A/Ro, and Ku;

DKK1, GRP78, SS-B/La, and Ku;

DKK1, ANGPTL4, GRP78, and SS-A/Ro;

DKKI, ANGPTL4, GRP78, and SS-B/La;

DKKI, ANGPTL4, GRP78, and Ku;

DKKI, ANGPTL4, GRP78, SS-A/Ro, and SS-B/La;

DKK1, ANGPTL4, GRP78, SS-A/Ro, and Ku;

DKK1, ANGPTL4, GRP78, SS-B/La, and Ku;

DKK1, MUC1, GRP78, and SS-A/Ro;

DKKI, MUC1, GRP78, and SS-B/La;

DKKI, MUC1, GRP78, and Ku;

DKKI, MUC1, GRP78, SS-A/Ro, and SS-B/La;

DKK1, MUC1, GRP78, SS-A/Ro, and Ku;

DKK1, MUC1, GRP78, SS-B/La, and Ku;

DKK1, ANGPTL4, MUC1, GRP78, and SS-A/Ro;

DKKI, ANGPTL4, MUC1, GRP78, and SS-B/La;

DKKI, ANGPTL4, MUC1, GRP78, and Ku;

DKKI, ANGPTL4, MUC1, GRP78, SS-A/Ro, and SS-B/La;

DKK1, ANGPTL4, MUC1, GRP78, SS-A/Ro, and Ku;

DKK1, ANGPTL4, MUC1, GRP78, SS-B/La, and Ku;

DKK1, GFRA1, and SS-A/Ro;

DKKI, GFRA1, and SS-B/La;

DKKI, GFRA1, and Ku;

DKKI, GFRA1, SS-A/Ro, and SS-B/La;

DKK1, GFRA1, SS-A/Ro, and Ku;

DKK1, GFRA1, SS-B/La, and Ku;

DKK1, ANGPTL4, GFRA1, and SS-A/Ro;

DKKI, ANGPTL4, GFRA1, and SS-B/La;

DKKI, ANGPTL4, GFRA1, and Ku;

DKKI, ANGPTL4, GFRA1, SS-A/Ro, and SS-B/La;

DKK1, ANGPTL4, GFRA1, SS-A/Ro, and Ku;

DKK1, ANGPTL4, GFRA1, SS-B/La, and Ku;

DKK1, MUC1, GFRA1, and SS-A/Ro;

DKKI, MUC1, GFRA1, and SS-B/La;

DKKI, MUC1, GFRA1, and Ku;

DKKI, MUC1, GFRA1, SS-A/Ro, and SS-B/La;

DKK1, MUC1, GFRA1, SS-A/Ro, and Ku;

DKK1, MUC1, GFRA1, SS-B/La, and Ku;

DKK1, ANGPTL4, MUC1, GFRA1, and SS-A/Ro;

DKKI, ANGPTL4, MUC1, GFRA1, and SS-B/La;

DKKI, ANGPTL4, MUC1, GFRA1, and Ku;

DKKI, ANGPTL4, MUC1, GFRA1, SS-A/Ro, and SS-B/La;

DKK1, ANGPTL4, MUC1, GFRA1, SS-A/Ro, and Ku;

DKK1, ANGPTL4, MUC1, GFRA1, SS-B/La, and Ku;

DKK1, ANGPTL4, MUC1, GRP78, GFRA1, and SS-A/Ro;

DKKI, ANGPTL4, MUC1, GRP78, GFRA1, and SS-B/La;

DKKI, ANGPTL4, MUC1, GRP78, GFRA1, and Ku;

DKKI, ANGPTL4, MUC1, GRP78, GFRA1, SS-A/Ro, and SS-B/La;

DKK1, ANGPTL4, MUC1, GRP78, GFRA1, SS-A/Ro, and Ku;

DKK1, ANGPTL4, MUC1, GRP78, GFRA1, SS-B/La, and Ku;

DKK1, A1AT, and SS-A/Ro;

DKKI, A1AT, and SS-B/La;

DKKI, A1AT, and Ku;

DKKI, A1AT, SS-A/Ro, and SS-B/La;

DKK1, A1AT, SS-A/Ro, and Ku;

DKK1, A1AT, SS-B/La, and Ku;

DKK1, ANGPTL4, A1AT, and SS-A/Ro;

DKKI, ANGPTL4, A1AT, and SS-B/La;

DKKI, ANGPTL4, A1AT, and Ku;

DKKI, ANGPTL4, A1AT, SS-A/Ro, and SS-B/La;

DKK1, ANGPTL4, A1AT, SS-A/Ro, and Ku;

DKK1, ANGPTL4, A1AT, SS-B/La, and Ku;

DKK1, MUC1, A1AT, and SS-A/Ro;

DKKI, MUC1, A1AT, and SS-B/La;

DKKI, MUC1, A1AT, and Ku;

DKKI, MUC1, A1AT, SS-A/Ro, and SS-B/La;

DKK1, MUC1, A1AT, SS-A/Ro, and Ku;

DKK1, MUC1, A1AT, SS-B/La, and Ku;

DKK1, ANGPTL4, MUC1, A1AT, and SS-A/Ro;

DKKI, ANGPTL4, MUC1, A1AT, and SS-B/La;

DKKI, ANGPTL4, MUC1, A1AT, and Ku;

DKKI, ANGPTL4, MUC1, A1AT, SS-A/Ro, and SS-B/La;

DKK1, ANGPTL4, MUC1, A1AT, SS-A/Ro, and Ku;

DKK1, ANGPTL4, MUC1, A1AT, SS-B/La, and Ku;

DKK1, ANGPTL4, MUC1, GRP78, A1AT, and SS-A/Ro;

DKKI, ANGPTL4, MUC1, GRP78, A1AT, and SS-B/La;

DKKI, ANGPTL4, MUC1, GRP78, A1AT, and Ku;

DKKI, ANGPTL4, MUC1, GRP78, A1AT, SS-A/Ro, and SS-B/La;

DKK1, ANGPTL4, MUC1, GRP78, A1AT, SS-A/Ro, and Ku;

DKK1, ANGPTL4, MUC1, GRP78, A1AT, SS-B/La, and Ku;

DKK1, ANGPTL4, MUC1, GRP78, GFRA1, A1AT, and SS-A/Ro;

DKKI, ANGPTL4, MUC1, GRP78, GFRA1, A1AT, and SS-B/La;

DKKI, ANGPTL4, MUC1, GRP78, GFRA1, A1AT, and Ku;

DKKI, ANGPTL4, MUC1, GRP78, GFRA1, A1AT, SS-A/Ro, and SS-B/La;

DKK1, ANGPTL4, MUC1, GRP78, GFRA1, A1AT, SS-A/Ro, and Ku;

DKK1, ANGPTL4, MUC1, GRP78, GFRA1, A1AT, SS-B/La, and Ku;

DKK1, GRN, and SS-A/Ro;

DKKI, GRN, and SS-B/La;

DKKI, GRN, and Ku;

DKKI, GRN, SS-A/Ro, and SS-B/La;

DKK1, GRN, SS-A/Ro, and Ku;

DKK1, GRN, SS-B/La, and Ku;

DKK1, ANGPTL4, GRN, and SS-A/Ro;

DKKI, ANGPTL4, GRN, and SS-B/La;

DKKI, ANGPTL4, GRN, and Ku;

DKKI, ANGPTL4, GRN, SS-A/Ro, and SS-B/La;

DKK1, ANGPTL4, GRN, SS-A/Ro, and Ku;

DKK1, ANGPTL4, GRN, SS-B/La, and Ku;

DKK1, MUC1, GRN, and SS-A/Ro;

DKKI, MUC1, GRN, and SS-B/La;

DKKI, MUC1, GRN, and Ku;

DKKI, MUC1, GRN, SS-A/Ro, and SS-B/La;

DKK1, MUC1, GRN, SS-A/Ro, and Ku;

DKK1, MUC1, GRN, SS-B/La, and Ku;

DKK1, ANGPTL4, MUC1, GRN, and SS-A/Ro;

DKKI, ANGPTL4, MUC1, GRN, and SS-B/La;

DKKI, ANGPTL4, MUC1, GRN, and Ku;

DKKI, ANGPTL4, MUC1, GRN, SS-A/Ro, and SS-B/La;

DKK1, ANGPTL4, MUC1, GRN, SS-A/Ro, and Ku;

DKK1, ANGPTL4, MUC1, GRN, SS-B/La, and Ku;

DKK1, ANGPTL4, MUC1, GRP78, GRN, and SS-A/Ro;

DKKI, ANGPTL4, MUC1, GRP78, GRN, and SS-B/La;

DKKI, ANGPTL4, MUC1, GRP78, GRN, and Ku;

DKKI, ANGPTL4, MUC1, GRP78, GRN, SS-A/Ro, and SS-B/La;

DKK1, ANGPTL4, MUC1, GRP78, GRN, SS-A/Ro, and Ku;

DKK1, ANGPTL4, MUC1, GRP78, GRN, SS-B/La, and Ku;

DKK1, ANGPTL4, MUC1, GRP78, GFRA1, GRN, and SS-A/Ro;

DKKI, ANGPTL4, MUC1, GRP78, GFRA1, GRN, and SS-B/La;

DKKI, ANGPTL4, MUC1, GRP78, GFRA1, GRN, and Ku;

DKKI, ANGPTL4, MUC1, GRP78, GFRA1, GRN, SS-A/Ro, and SS-B/La;

DKK1, ANGPTL4, MUC1, GRP78, GFRA1, GRN, SS-A/Ro, and Ku;

DKK1, ANGPTL4, MUC1, GRP78, GFRA1, GRN, SS-B/La, and Ku;

DKK1, ANGPTL4, MUC1, GRP78, GFRA1, A1AT, GRN, and SS-A/Ro;

DKKI, ANGPTL4, MUC1, GRP78, GFRA1, A1AT, GRN, and SS-B/La;

DKKI, ANGPTL4, MUC1, GRP78, GFRA1, A1AT, GRN, and Ku;

DKKI, ANGPTL4, MUC1, GRP78, GFRA1, A1AT, GRN, SS-A/Ro, and SS-B/La;

DKK1, ANGPTL4, MUC1, GRP78, GFRA1, A1AT, GRN, SS-A/Ro, and Ku;

DKK1, ANGPTL4, MUC1, GRP78, GFRA1, A1AT, GRN, SS-B/La, and Ku;

ANGPTL4 and SS-A/Ro;

ANGPTL4 and SS-B/La;

ANGPTL4 and Ku;

ANGPTL4, SS-A/Ro, and SS-B/La;

ANGPTL4, SS-A/Ro, and Ku;

ANGPTL4, SS-B/La, and Ku;

ANGPTL4, MUC1, and SS-A/Ro;

ANGPTL4, MUC1, and SS-B/La;

ANGPTL4, MUC1, and Ku;

ANGPTL4, MUC1, SS-A/Ro, and SS-B/La;

ANGPTL4, MUC1, SS-A/Ro, and Ku;

ANGPTL4, MUC1, SS-B/La, and Ku;

ANGPTL4, GRP78, and SS-A/Ro;

ANGPTL4, GRP78, and SS-B/La;

ANGPTL4, GRP78, and Ku;

ANGPTL4, GRP78, SS-A/Ro, and SS-B/La;

ANGPTL4, GRP78, SS-A/Ro, and Ku;

ANGPTL4, GRP78, SS-B/La, and Ku;

ANGPTL4, MUC1, GRP78, and SS-A/Ro;

ANGPTL4, MUC1, GRP78, and SS-B/La;

ANGPTL4, MUC1, GRP78, and Ku;

ANGPTL4, MUC1, GRP78, SS-A/Ro, and SS-B/La;

ANGPTL4, MUC1, GRP78, SS-A/Ro, and Ku;

ANGPTL4, MUC1, GRP78, SS-B/La, and Ku;

ANGPTL4, GFRA1, and SS-A/Ro;

ANGPTL4, GFRA1, and SS-B/La;

ANGPTL4, GFRA1, and Ku;

ANGPTL4, GFRA1, SS-A/Ro, and SS-B/La;

ANGPTL4, GFRA1, SS-A/Ro, and Ku;

ANGPTL4, GFRA1, SS-B/La, and Ku;

ANGPTL4, MUC1, GFRA1, and SS-A/Ro;

ANGPTL4, MUC1, GFRA1, and SS-B/La;

ANGPTL4, MUC1, GFRA1, and Ku;

ANGPTL4, MUC1, GFRA1, SS-A/Ro, and SS-B/La;

ANGPTL4, MUC1, GFRA1, SS-A/Ro, and Ku;

ANGPTL4, MUC1, GFRA1, SS-B/La, and Ku;

ANGPTL4, MUC1, GRP78, GFRA1, and SS-A/Ro;

ANGPTL4, MUC1, GRP78, GFRA1, and SS-B/La;

ANGPTL4, MUC1, GRP78, GFRA1, and Ku;

ANGPTL4, MUC1, GRP78, GFRA1, SS-A/Ro, and SS-B/La;

ANGPTL4, MUC1, GRP78, GFRA1, SS-A/Ro, and Ku;

ANGPTL4, MUC1, GRP78, GFRA1, SS-B/La, and Ku;

ANGPTL4, A1AT, and SS-A/Ro;

ANGPTL4, A1AT, and SS-B/La;

ANGPTL4, A1AT, and Ku;

ANGPTL4, A1AT, SS-A/Ro, and SS-B/La;

ANGPTL4, A1AT, SS-A/Ro, and Ku;

ANGPTL4, A1AT, SS-B/La, and Ku;

ANGPTL4, MUC1, A1AT, and SS-A/Ro;

ANGPTL4, MUC1, A1AT, and SS-B/La;

ANGPTL4, MUC1, A1AT, and Ku;

ANGPTL4, MUC1, A1AT, SS-A/Ro, and SS-B/La;

ANGPTL4, MUC1, A1AT, SS-A/Ro, and Ku;

ANGPTL4, MUC1, A1AT, SS-B/La, and Ku;

ANGPTL4, MUC1, GRP78, A1AT, and SS-A/Ro;

ANGPTL4, MUC1, GRP78, A1AT, and SS-B/La;

ANGPTL4, MUC1, GRP78, A1AT, and Ku;

ANGPTL4, MUC1, GRP78, A1AT, SS-A/Ro, and SS-B/La;

ANGPTL4, MUC1, GRP78, A1AT, SS-A/Ro, and Ku;

ANGPTL4, MUC1, GRP78, A1AT, SS-B/La, and Ku;

ANGPTL4, MUC1, GRP78, GFRA1, A1AT, and SS-A/Ro;

ANGPTL4, MUC1, GRP78, GFRA1, A1AT, and SS-B/La;

ANGPTL4, MUC1, GRP78, GFRA1, A1AT, and Ku;

ANGPTL4, MUC1, GRP78, GFRA1, A1AT, SS-A/Ro, and SS-B/La;

ANGPTL4, MUC1, GRP78, GFRA1, A1AT, SS-A/Ro, and Ku;

ANGPTL4, MUC1, GRP78, GFRA1, A1AT, SS-B/La, and Ku;

ANGPTL4, GRN, and SS-A/Ro;

ANGPTL4, GRN, and SS-B/La;

ANGPTL4, GRN, and Ku;

ANGPTL4, GRN, SS-A/Ro, and SS-B/La;

ANGPTL4, GRN, SS-A/Ro, and Ku;

ANGPTL4, GRN, SS-B/La, and Ku;

ANGPTL4, MUC1, GRN, and SS-A/Ro;

ANGPTL4, MUC1, GRN, and SS-B/La;

ANGPTL4, MUC1, GRN, and Ku;

ANGPTL4, MUC1, GRN, SS-A/Ro, and SS-B/La;

ANGPTL4, MUC1, GRN, SS-A/Ro, and Ku;

ANGPTL4, MUC1, GRN, SS-B/La, and Ku;

ANGPTL4, MUC1, GRP78, GRN, and SS-A/Ro;

ANGPTL4, MUC1, GRP78, GRN, and SS-B/La;

ANGPTL4, MUC1, GRP78, GRN, and Ku;

ANGPTL4, MUC1, GRP78, GRN, SS-A/Ro, and SS-B/La;

ANGPTL4, MUC1, GRP78, GRN, SS-A/Ro, and Ku;

ANGPTL4, MUC1, GRP78, GRN, SS-B/La, and Ku;

ANGPTL4, MUC1, GRP78, GFRA1, GRN, and SS-A/Ro;

ANGPTL4, MUC1, GRP78, GFRA1, GRN, and SS-B/La;

ANGPTL4, MUC1, GRP78, GFRA1, GRN, and Ku;

ANGPTL4, MUC1, GRP78, GFRA1, GRN, SS-A/Ro, and SS-B/La;

ANGPTL4, MUC1, GRP78, GFRA1, GRN, SS-A/Ro, and Ku;

ANGPTL4, MUC1, GRP78, GFRA1, GRN, SS-B/La, and Ku;

ANGPTL4, MUC1, GRP78, GFRA1, A1AT, GRN, and SS-A/Ro;

ANGPTL4, MUC1, GRP78, GFRA1, A1AT, GRN, and SS-B/La;

ANGPTL4, MUC1, GRP78, GFRA1, A1AT, GRN, and Ku;

ANGPTL4, MUC1, GRP78, GFRA1, A1AT, GRN, SS-A/Ro, and SS-B/La;

ANGPTL4, MUC1, GRP78, GFRA1, A1AT, GRN, SS-A/Ro, and Ku;

ANGPTL4, MUC1, GRP78, GFRA1, A1AT, GRN, SS-B/La, and Ku;

MUC1 and SS-A/Ro;

MUC1 and SS-B/La;

MUC1 and Ku;

MUC1, SS-A/Ro, and SS-B/La;

MUC1, SS-A/Ro, and Ku;

MUC1, SS-B/La, and Ku;

MUC1, GRP78, and SS-A/Ro;

MUC1, GRP78, and SS-B/La;

MUC1, GRP78, and Ku;

MUC1, GRP78, SS-A/Ro, and SS-B/La;

MUC1, GRP78, SS-A/Ro, and Ku;

MUC1, GRP78, SS-B/La, and Ku;

MUC1, GFRA1, and SS-A/Ro;

MUC1, GFRA1, and SS-B/La;

MUC1, GFRA1, and Ku;

MUC1, GFRA1, SS-A/Ro, and SS-B/La;

MUC1, GFRA1, SS-A/Ro, and Ku;

MUC1, GFRA1, SS-B/La, and Ku;

MUC1, GRP78, GFRA1, and SS-A/Ro;

MUC1, GRP78, GFRA1, and SS-B/La;

MUC1, GRP78, GFRA1, and Ku;

MUC1, GRP78, GFRA1, SS-A/Ro, and SS-B/La;

MUC1, GRP78, GFRA1, SS-A/Ro, and Ku;

MUC1, GRP78, GFRA1, SS-B/La, and Ku;

MUC1, A1AT, and SS-A/Ro;

MUC1, A1AT, and SS-B/La;

MUC1, A1 AT, and Ku;

MUC1, A1AT, SS-A/Ro, and SS-B/La;

MUC1, A1AT, SS-A/Ro, and Ku;

MUC1, A1AT, SS-B/La, and Ku;

MUC1, GRP78, A1AT, and SS-A/Ro;

MUC1, GRP78, A1AT, and SS-B/La;

MUC1, GRP78, A1AT, and Ku;

MUC1, GRP78, A1AT, SS-A/Ro, and SS-B/La;

MUC1, GRP78, A1AT, SS-A/Ro, and Ku;

MUC1, GRP78, A1AT, SS-B/La, and Ku;

MUC1, GRP78, GFRA1, A1AT, and SS-A/Ro;

MUC1, GRP78, GFRA1, A1AT, and SS-B/La;

MUC1, GRP78, GFRA1, A1AT, and Ku;

MUC1, GRP78, GFRA1, A1AT, SS-A/Ro, and SS-B/La;

MUC1, GRP78, GFRA1, A1AT, SS-A/Ro, and Ku;

MUC1, GRP78, GFRA1, A1AT, SS-B/La, and Ku;

MUC1, GRN, and SS-A/Ro;

MUC1, GRN, and SS-B/La;

MUC1, GRN, and Ku;

MUC1, GRN, SS-A/Ro, and SS-B/La;

MUC1, GRN, SS-A/Ro, and Ku;

MUC1, GRN, SS-B/La, and Ku;

MUC1, GRP78, GRN, and SS-A/Ro;

MUC1, GRP78, GRN, and SS-B/La;

MUC1, GRP78, GRN, and Ku;

MUC1, GRP78, GRN, SS-A/Ro, and SS-B/La;

MUC1, GRP78, GRN, SS-A/Ro, and Ku;

MUC1, GRP78, GRN, SS-B/La, and Ku;

MUC1, GRP78, GFRA1, GRN, and SS-A/Ro;

MUC1, GRP78, GFRA1, GRN, and SS-B/La;

MUC1, GRP78, GFRA1, GRN, and Ku;

MUC1, GRP78, GFRA1, GRN, SS-A/Ro, and SS-B/La;

MUC1, GRP78, GFRA1, GRN, SS-A/Ro, and Ku;

MUC1, GRP78, GFRA1, GRN, SS-B/La, and Ku;

MUC1, GRP78, GFRA1, A1AT, GRN, and SS-A/Ro;

MUC1, GRP78, GFRA1, A1AT, GRN, and SS-B/La;

MUC1, GRP78, GFRA1, A1AT, GRN, and Ku;

MUC1, GRP78, GFRA1, A1AT, GRN, SS-A/Ro, and SS-B/La;

MUC1, GRP78, GFRA1, A1AT, GRN, SS-A/Ro, and Ku;

MUC1, GRP78, GFRA1, A1AT, GRN, SS-B/La, and Ku;

GRP78 and SS-A/Ro;

GRP78 and SS-B/La;

GRP78 and Ku;

GRP78, SS-A/Ro, and SS-B/La;

GRP78, SS-A/Ro, and Ku;

GRP78, SS-B/La, and Ku;

GRP78, GFRA1, and SS-A/Ro;

GRP78, GFRA1, and SS-B/La;

GRP78, GFRA1, and Ku;

GRP78, GFRA1, SS-A/Ro, and SS-B/La;

GRP78, GFRA1, SS-A/Ro, and Ku;

GRP78, GFRA1, SS-B/La, and Ku;

GRP78, A1AT, and SS-A/Ro;

GRP78, A1AT, and SS-B/La;

GRP78, A1AT, and Ku;

GRP78, A1AT, SS-A/Ro, and SS-B/La;

GRP78, A1AT, SS-A/Ro, and Ku;

GRP78, A1AT, SS-B/La, and Ku;

GRP78, GFRA1, A1AT, and SS-A/Ro;

GRP78, GFRA1, A1AT, and SS-B/La;

GRP78, GFRA1, A1AT, and Ku;

GRP78, GFRA1, A1AT, SS-A/Ro, and SS-B/La;

GRP78, GFRA1, A1AT, SS-A/Ro, and Ku;

GRP78, GFRA1, A1AT, SS-B/La, and Ku;

GRP78, GRN, and SS-A/Ro;

GRP78, GRN, and SS-B/La;

GRP78, GRN, and Ku;

GRP78, GRN, SS-A/Ro, and SS-B/La;

GRP78, GRN, SS-A/Ro, and Ku;

GRP78, GRN, SS-B/La, and Ku;

GRP78, GFRA1, GRN, and SS-A/Ro;

GRP78, GFRA1, GRN, and SS-B/La;

GRP78, GFRA1, GRN, and Ku;

GRP78, GFRA1, GRN, SS-A/Ro, and SS-B/La;

GRP78, GFRA1, GRN, SS-A/Ro, and Ku;

GRP78, GFRA1, GRN, SS-B/La, and Ku;

GRP78, GFRA1, A1AT, GRN, and SS-A/Ro;

GRP78, GFRA1, A1AT, GRN, and SS-B/La;

GRP78, GFRA1, A1AT, GRN, and Ku;

GRP78, GFRA1, A1AT, GRN, SS-A/Ro, and SS-B/La;

GRP78, GFRA1, A1AT, GRN, SS-A/Ro, and Ku;

GRP78, GFRA1, A1AT, GRN, SS-B/La, and Ku;

GFRA1 and SS-A/Ro;

GFRA1 and SS-B/La;

GFRA1 and Ku;

GFRA1, SS-A/Ro, and SS-B/La;

GFRA1, SS-A/Ro, and Ku;

GFRA1, SS-B/La, and Ku;

GFRA1, A1AT, and SS-A/Ro;

GFRA1, A1AT, and SS-B/La;

GFRA1, A1AT, and Ku;

GFRA1, A1AT, SS-A/Ro, and SS-B/La;

GFRA1, A1AT, SS-A/Ro, and Ku;

GFRA1, A1AT, SS-B/La, and Ku;

GFRA1, GRN, and SS-A/Ro;

GFRA1, GRN, and SS-B/La;

GFRA1, GRN, and Ku;

GFRA1, GRN, SS-A/Ro, and SS-B/La;

GFRA1, GRN, SS-A/Ro, and Ku;

GFRA1, GRN, SS-B/La, and Ku;

GFRA1, A1AT, GRN, and SS-A/Ro;

GFRA1, A1AT, GRN, and SS-B/La;

GFRA1, A1AT, GRN, and Ku;

GFRA1, A1AT, GRN, SS-A/Ro, and SS-B/La;

GFRA1, A1AT, GRN, SS-A/Ro, and Ku;

GFRA1, A1AT, GRN, SS-B/La, and Ku;

A1AT and SS-A/Ro;

A1AT and SS-B/La;

A1AT and Ku;

A1AT, SS-A/Ro, and SS-B/La;

A1AT, SS-A/Ro, and Ku;

A1AT, SS-B/La, and Ku;

A1AT, GRN, and SS-A/Ro;

A1AT, GRN, and SS-B/La;

A1AT, GRN, and Ku;

A1AT, GRN, SS-A/Ro, and SS-B/La;

A1AT, GRN, SS-A/Ro, and Ku; and

A1AT, GRN, SS-B/La, and Ku.

In another embodiment, the one or more first antibody detection marker molecules comprise or consist of the TAA, an antigenic fragment thereof, or a native extracellular domain and/or native secreted version of the TAA or antigenic fragment thereof and the one or more second antibody detection marker molecules comprise or consist of the ENA or antigenic fragment thereof, or a native extracellular domains and/or native secreted version of the ENA or antigenic fragment thereof.

In a further embodiment, the composition consists of between 2-10,000 antibody detection marker molecules in total. In another embodiment, the composition consists of between 2 and 1000 antibody detection marker molecules in total.

### EXAMPLES

### Example/Case Study 1

ANA staining was performed on sera samples collected from 200 newly diagnosed breast cancer patients before the start of any treatment. This patient population represented the heterogeneity of the disease consisting of 74% invasive, 26% in situ, 87% ductal, 11% lobular, 24% lymph node involvement, 86% ER positive, 17% HER-2 positive, and 12% triple negative breast cancer. HEp-2 cells were seeded in 96-well tissue culture plates. The cells were fixed, permeabilized and incubated with serum samples at an initial dilution of 1:40. The secondary antibody, Alexa™ 488 goat anti-human IgG, was added for pattern detection. The cells were imaged with the Celllnsight CX7™ instrument. Positive samples at the 1:40 dilution were further screened with a 1:2 serial dilution of 1:40, 1:80, 1:160 and 1:320. Two scientists independently scored the staining patterns to ensure accurate and reproducible pattern determination. Reference serum for each staining pattern was acquired from SLR Research Corporation, Carlsbad, Calif.

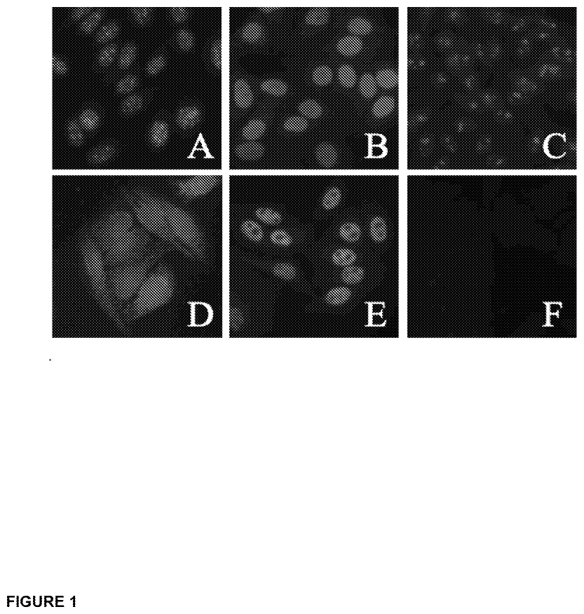

For a positive ANA test, both a defined staining pattern and dilution are reported. FIG. 1 shows examples generated of the different positive staining patterns: homogeneous, speckled, centromeric, mitochondrial and nucleolar. Sera used in these assays were from cancer patients in the studied population without diagnosed autoimmune disease. For ANA assays, staining at a 1:160 dilution is considered positive. Serum titrations are essential for ANA testing because 32% of healthy individuals will be positive at a 1:40 serum dilution. However, at 1:80, 13.3% of individuals are positive, 5.0% at 1:160 and only 3.3% at 1:320 [9]. The results indicated that 36.5% of the patients were positive for ANA staining at a 1:160 dilution. Additionally, breast cancer subtypes did not correlate with a specific staining pattern. Table 1 indicates the number of breast cancer patients that had positive staining patterns for centromeric, homogenous, nucleolar, mitochondrial and speckled, verifying that cancer patients have autoimmune antibodies.

TABLE 1
ANA Staining pattern of 200 breast cancer patients
ANA

| Staining | | | | | | Total | No |
|---|---|---|---|---|---|---|---|
| pattern | Centromeric | Homogenous | Nucleolar | Mitochondrial | Speckled | positive* | staining |
| # of | 1 | 33 | 16 | 4 | 19 | 73 | 127 |
| patients + | | | | | | | |
| staining | | | | | | | |
| % of 200 | 0.5% | 16.5% | 8% | 2% | 9.5% | 36.5% | 63.5% |

*% positive ANA staining at 1:160 dilution of 200 breast cancer patients

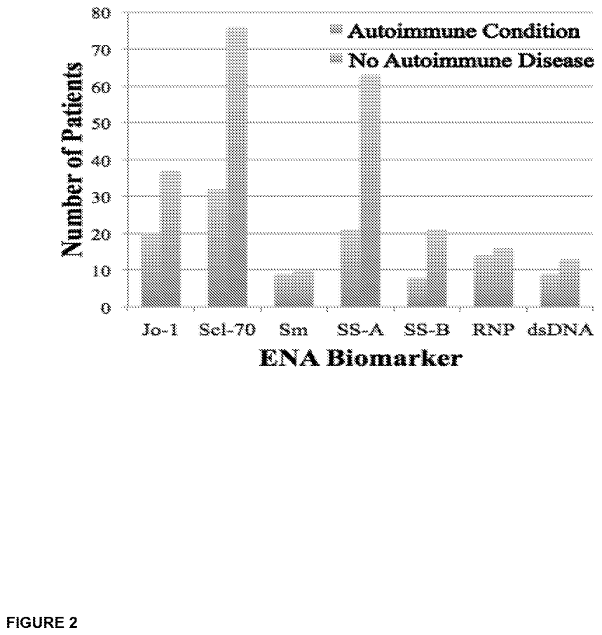

It was further determined how many of the 200 breast cancer patients from the cohort described above do not have diagnosed autoimmune disease, but still have anti-ENA autoantibodies. The serum collected from 200 breast cancer patients before the start of treatment was surveyed for reactivity against a panel of ENA autoimmune biomarkers: Jo-1, Ku, SS-A (Ro-60 kDa), SS-A (Ro-52 kDa), SS-B (La), Scl-70, Sm, SmD1 peptide, RNP, U1-snRNP, PM/Scl75, dsDNA, CENP-A and CENP-B. A Luminex bead-based multiplex assay was used to detect autoantibody reactivity against the ENA proteins (Table 2 and FIG. 2). The Luminex xMAP™ microspheres technology (Luminex, Austin, Tex.) is based on color-coded, 5.6 micron beads, called microspheres, which are dyed with distinct fluorophores and used as the solid surface on which to build the assay. Each of the ENA proteins was bound to a different colored Luminex bead. An equal mixture of the ENA antigen-coated bead sets were placed in a single tube and incubated with serum. An anti-human IgG antibody conjugated with PE was utilized as the secondary antibody. For each individual bead, the Luminex system measures the fluorescence of the internal dye to identify the bead and PE to determine autoantibody response until at least 100 of each bead color have been analyzed, allowing for statistical power. Reference antisera against the ENA markers was acquired from the Centers for Disease Control (CDC) and used as positive controls.

As a representation of the generated data, results from 7 ENA biomarkers are shown in Table 2. A positive anti-ENA antibody response is defined as 10-fold greater reactivity over background. The data confirmed that a set of patients have autoantibodies against ENA autoimmune proteins even though they do not have diagnosed autoimmune disease prior to cancer diagnosis. For example, 57 of 200 patients were positive for autoantibodies against Jo-1 (Table 3). Of the 57 Jo-1 positive patients, 20 had autoimmune disease. However, 37 patients were serotype positive, but asymptomatic for autoimmune disease (graphed in FIG. 2). This result clearly indicates that cancer patients have autoantibodies against ENA autoimmune proteins even though they have not been clinically diagnosed with outright autoimmune disease.

TABLE 2
Classification of patient autoimmune condition with responses against ENA
biomarkers.

| | Jo-1 | | Scl-70 | | SS-A | | SS-B | | Sm | | RNP | | dsDNA | |
|---|---|---|---|---|---|---|---|---|---|---|---|---|---|---|
| ENA | + | − | + | − | + | − | + | − | + | − | + | − | + | − |
| Total patients | 57 | 143 | 108 | 92 | 84 | 116 | 29 | 171 | 19 | 181 | 30 | 170 | 22 | 178 |
| (n = 200) | | | | | | | | | | | | | | |
| Autoimmune | 20 | 32 | 32 | 20 | 21 | 31 | 8 | 44 | 9 | 43 | 14 | 38 | 9 | 43 |
| condition (n = 52) | | | | | | | | | | | | | | |
| No autoimmune | 37 | 111 | 76 | 72 | 63 | 85 | 21 | 127 | 10 | 138 | 16 | 132 | 13 | 135 |
| condition (n = 148) | | | | | | | | | | | | | | |

### Example/Case Study 2

### Overview

Bio-ID™ cartridges were produced in-house and contained a multiplex array of capture antigens for the following biomarker candidates:

### TAA's: ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, AIAT, BIRC5, GAL3, CAPC, GRP78.

ENA's: CENPA, Jo-1, RNP/Sm, Scl70, Sm, SS-A/Ro (52 kDa), SS-B/La, CENPB, U1-snRNP, PM/Scl100, SS-A/Ro (60 kDa), Ku, PM/Scl75.

All antigens plus controls and standards were printed in duplicate onto the Bio-ID™ cartridges using an out-sourced microarray printing facility (Applied Microarrays (AMI)). Several assay parameters were optimized to minimize assay to assay variation and maximize biochip performance. This included: optimizing antigen spotting concentration, number of droplets per spot, drying conditions, detection antibody concentration, and antibody incubation times. Standard diluents were used to assess reagent compatibility, signal control technical replicates were used to assess assay to assay variation, and reference samples were purchased from SLR Research to validate dilution linearity and detection range.

### Experimental Approach and Results

The starting point for the assay development work was to optimize the arraying/printing process with AMI. Spot quality (spot morphology and spot size variation) was used to evaluate and optimize initial arraying conditions. Bio-ID™ slides were produced in-house and shipped to AMI for arraying with a set of control content (BC biomarkers with rFc tags). AMI proceeded to print small batches of test slides which were shipped back to our laboratory in Sioux Falls, where the printed slides were bonded with the Bio-ID™ PDMS caps (to make them compatible with the Bio-ID™ analyzer). The cartridges were processed with conjugated goat anti-rabbit IgG Dyelight™ 550 detection reagent known to interact with the rFc tag. All rFc tagged antigens picked up the signal to provide clear insight into spot morphology and size.

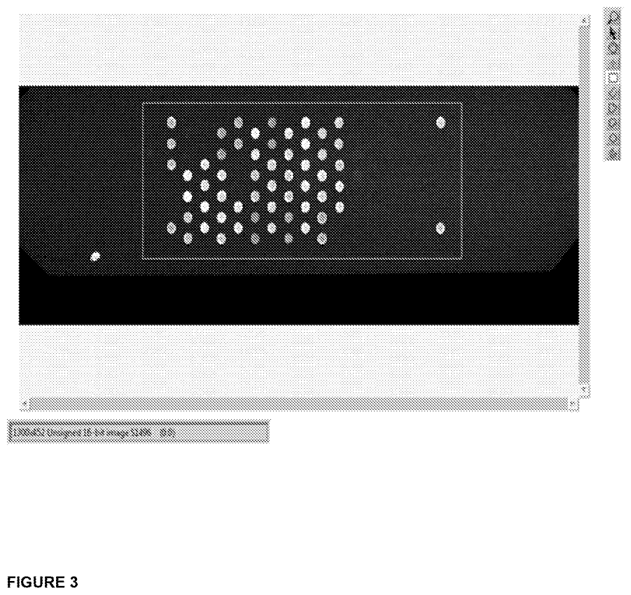

Through an iterative ‘print, run, evaluate process’, print conditions (print concentration, print buffer, number of drops per spot, drying conditions) were varied to optimize spot shape and size. Detection antibody concentration and incubation time were also varied to optimize signal quality/strength. The resultant optimized print and processing conditions were then carried through to the full context multiplex arraying process. FIG. 3 shows an example image of the excellent spot morphology and spot size variation achieved through this process.

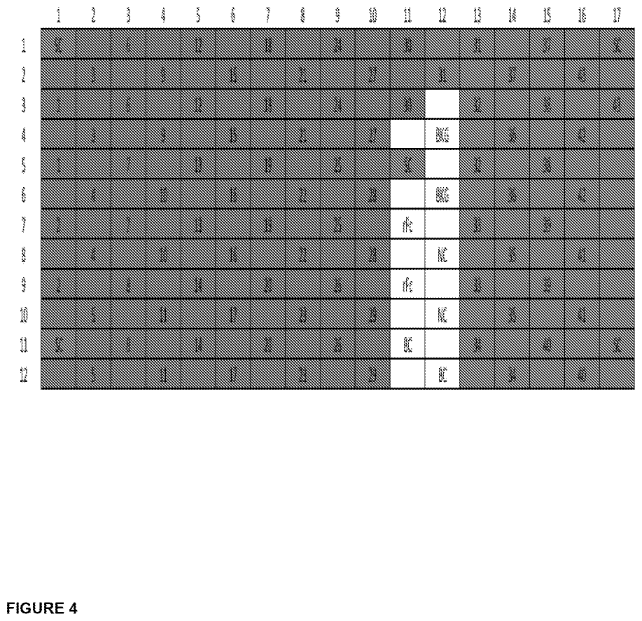

Following this initial development work, AMI proceeded to print further slides with the full 43-plex biomarker assay. As with the printing optimization process, these slides were bonded with PDMS caps and stored ready for use for assay validation and sample trials. The final layout of the 43-plex cartridges alongside a summary of the optimized print and process conditions in provided in FIG. 4.

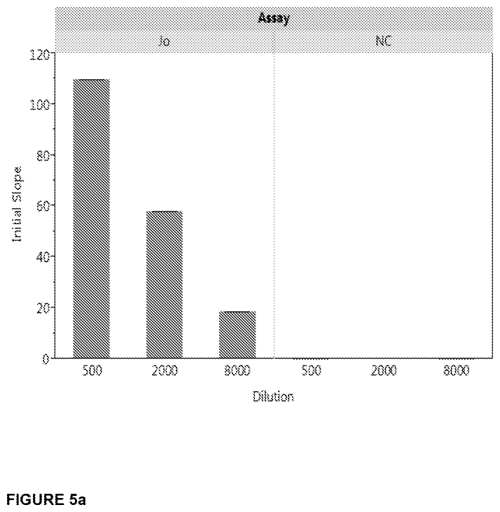

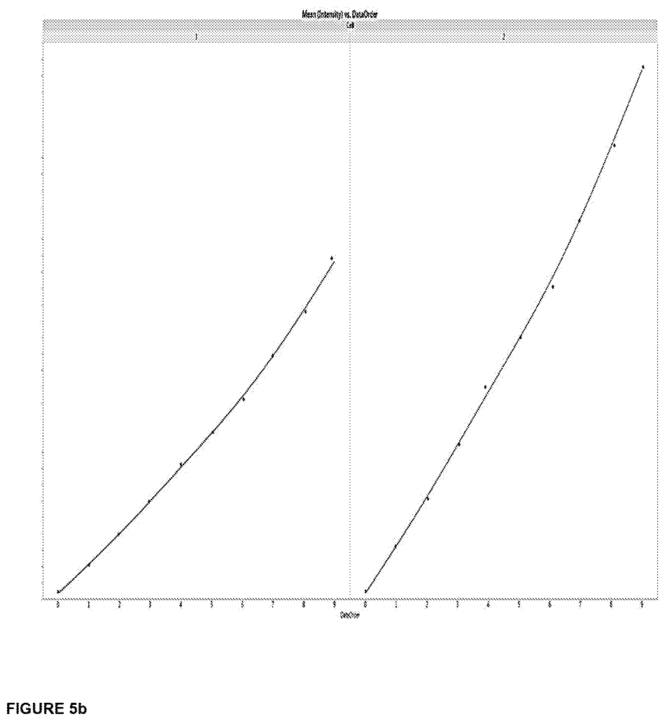

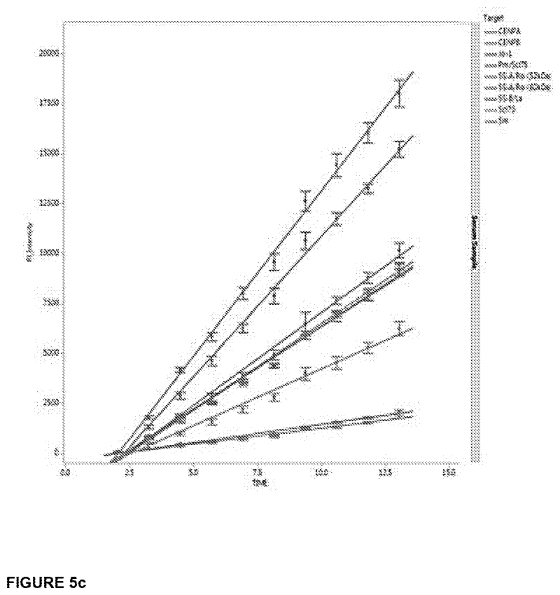

Prior to advancing into the sample runs, additional assay validation experiments were completed to ensure the assays were meeting targeted performance metrics of assay to assay CV's<15%, and semi-quantitative concentration dependent signals (dilution linearity). This was completed by processing AMLI Consensus Reference Panel serum samples purchased from SLR Research (using the optimized print and processing conditions). These serum samples are well characterized reference samples qualified to contain defined levels of one or more of the ENA autoimmune biomarkers included in the 43-plex assay. Reference sera were tested singly and in combinations. This work and data demonstrated an accurate dose-proportional response on biomarker concentration (dilution linearity) for all reference serum tested (reference sera qualified for the ENA markers CENPA, CENPB, Jo-1, Pm/Scl75, SSA(Ro52), SSA(Ro60), SSB(La), Scl70 and Sm were tested singly or in combinations across the multiplex). FIGS. 5a and 5 show example data. To further validate the Bio-ID's capability to detect across a clinically relevant range for our multiplex, a combination of all Reference sera was run including low titers of Jo-1 and SS-B (high concentration) and high titers of CENPA and Scl75 (low concentration). All reference sera were positively detected in a single run (see FIG. 5c). The Bio-ID's detection range was further validated through the patient sample studies wherein all biomarkers were detectable using a single sample dilution.

The final component of assay validation was to assess assay to assay Coefficient of Variability (CV). This was measured by processing the same reference serum sample across 26 separate assays (we used the SSA reference sample and measured the response of the SSA biomarker in our 43-plex panel). The mean value and standard deviation of each signal (measured as the gradient of the slope of the time-course signal plots generated by the Bio-ID) were calculated across all assays (n=26). The CV was then calculated as the standard deviation divided by the mean multiplied by 100. This figure represents a measure of the reproducibility of our system from one test to another. The result was an assay to assay CV of 8.06%. (see Table 3).

TABLE 3
Data from Assay to Assay Coefficient of Variability (CV) studies completed on the
Bio-ID 40-plex.
Reference Serum for SSA

| n | 1 | 2 | 3 | 4 | 5 | 6 | 7 | 8 | 9 | 10 | 11 | 12 | 13 |
|---|---|---|---|---|---|---|---|---|---|---|---|---|---|
| signal | 0.804 | 0.821 | 0.711 | 0.665 | 0.676 | 0.629 | 0.91 | 0.851 | 0.783 | 0.772 | 0.724 | 0.727 | 0.798 |
| n | 14 | 15 | 16 | 17 | 18 | 19 | 20 | 21 | 22 | 23 | 24 | 25 | 26 |
| signal | 0.697 | 0.757 | 0.769 | 0.749 | 0.72 | 0.694 | 0.711 | 0.757 | 0.734 | 0.747 | 0.755 | 0.739 | 0.688 |

| | | |
|---|---|---|
| Mean = 0.746 | Standard = Deviation 0.0601 | CV = 8.06% |

### Breast Cancer Sample Studies

Following completion of assay development and optimization, 200 BC samples and 200 age matched healthy controls were processed across the 43-plex breast cancer multiplex assay and the performance of the assay to discriminate BC from healthy samples was assessed.

The Bio-ID™ system was primed using filtered PBS buffer before initiating the assay procedure. The test cartridges (containing the 43-plex assay) undergo an initial hydration step, wherein a proprietary buffer is flowed across the multiplex to hydrate the capture agent spots and ready the cartridge for sample processing. The Bio-ID™ input wells were then loaded with patient samples at 1:50 dilutions (odd numbered channels) and secondary detection antibody (even numbered channels). The sample diluent was prepared by mixing 1% BSA, 5% Seablock™ and 0.1% tween-20 in PBS buffer. The secondary detection antibody used was Anti-Human IgG (H&L) (GOAT) Antibody DY550 labelled at 2 ug/ml. The antibody diluent was prepared by using 50% seablock and 0.1% tween-20 in PBS buffer. Sample and secondary detection antibody were then flowed iteratively across the multiplex, and the fluorescent intensity of each assay spot was measured following each cycle. A total of 14 cycles were performed through the course of the assay run, generating a 14 data point curve.

The resultant data from all 400 assay runs was uploaded to an analysis package (JMP) for statistical processing. In advance of the more quantitative analysis an initial qualitative assessment of the data was also made by comparing individual samples against their aged matched healthy controls, and assessing whether a clear difference across one or more biomarkers was present between the BC sample and the control.

This initial qualitative assessment demonstrated clear differentiation across a significant range of biomarkers in the 43-plex panel. This initial analysis was further supplemented by a qualitative meta-analysis of all BC samples against all normal controls processed to date. This data (not shown) highlights those biomarkers presenting consistent elevation across all the BC samples processed to date. While individual patient differences are to be expected in a test assessing auto-immune response, the fact that 16 biomarkers are consistently raised across the BC sample cohort is a clear indicator of a high population wide predictive value. Furthermore, the 16 ‘high discrimination markers’ contain a mix of both ENA and TAA markers, further demonstrates the significant utility of this novel testing approach for cancer detection (including diagnosis, prognosis and/or monitoring).

### Quantitative Analysis and Results

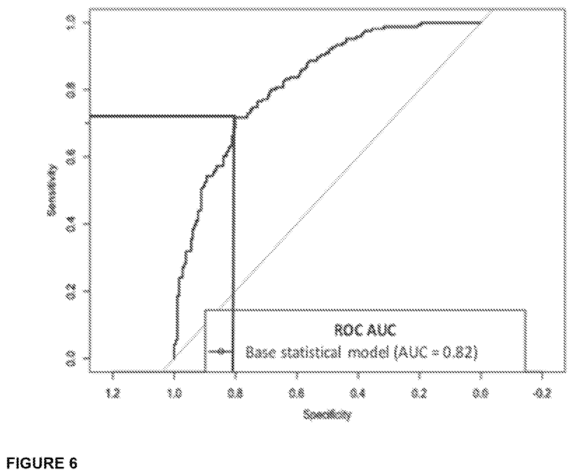

To quantify the discriminatory power of the expanded multiplex, a logistic regression model was used to test the contribution of each biomarker to the c-statistic (i.e., area under the ROC curve) and multivariable conditional logistic regression analysis was used to determine which biomarkers had the highest predictive value for discriminating healthy from cancerous samples in a single test. The result of this analysis is an AUC of 0.82. FIG. 6 summarizes ROC curve data and results from this quantitative analysis. The biomarkers identified as having the highest predictive value for discriminating healthy from cancerous samples in a single test using this model are listed in Table 4.

TABLE 4
The biomarkers with highest predictive value for
discriminating healthy from cancerous
samples in a single test.
The high predictive set from this data run contains
4 ENA markers, supporting the thesis that
standard auto-immune markers can be used to
strengthen overall predictive power of a diagnostic
test when coupled with a panel of TAA's.

| Table 4: High predictive markers | | | |
|---|---|---|---|
| | Name | Marker Type | Pr(>\|z\|) |
| 1 | SS-A/Ro | ENA | 0.000495 |
| 2 | KU | ENA | 0.000518 |
| 3 | SS-B/La | ENA | 0.000838 |
| 4 | LRRC15 | Bca | 0.001655 |
| 5 | CALD1 | Bca | 0.002131 |
| 6 | SSR2 | Bca | 0.014374 |
| 7 | MUC1 | Bca | 0.018078 |
| 8 | SPON2 | Bca | 0.027872 |
| 9 | GRP78 | Bca | 0.086227 |
| 10 | ANGPTL4 | Bca | 0.133549 |
| 11 | CDH3 | Bca | 0.169455 |
| 12 | CENPB | ENA | 0.189462 |
| 13 | GFRA1 | Bca | 0.271019 |
| 14 | SPINT2 | Bca | 0.377648 |
| 15 | AIAT | ENA | 0.475853 |
| 16 | DKK1 | Bca | 0.543908 |
| 17 | GRN | Bca | 0.713494 |
| 18 | CD320 | Bca | 0.90851 |

1. A method for diagnosing cancer, prognosing cancer occurrence or recurrence, and/or monitoring cancer therapy, comprising contacting a bodily fluid sample from a subject at risk of having cancer or cancer recurrence, or from a subject that has been treated for cancer with;
(a) one or more first antibody detection marker molecules that bind to human autoantibodies against at least one tumor associated antigen (TAA); and
(b) one or more second antibody detection marker molecules that bind to human autoantibodies against at least one extractable nuclear antigen (ENA);
wherein the presence of autoantibodies against one or more TAA and one or more ENA indicate a likelihood of the subject having cancer or cancer recurrence, and/or indicates efficacy of cancer therapy.

2.-22. (canceled)

23. An antibody detection marker composition comprising
(a) one or more first antibody detection marker molecules that bind to human autoantibodies against at least one tumor associated antigen (TAA); and
(b) one or more second antibody detection marker molecules that bind to human autoantibodies against at least one extractable nuclear antigen (ENA).

24. The composition of claim 23, wherein the one or more ENA is selected from the group consisting of CENPA, Jo-1, U1-snRNP subunit 70 kD, U1-snRNP subunit A, U1-snRNP subunit C, Sm subunit B, Sm subunit B′, Sm subunit D, Sm subunit E, Sm subunit F, Sm subunit G, Scl70, Sm, SS-A/Ro (52 kDa), SS-B/La, CENPB, U1-snRNP, PM/Scl100, SS-A/Ro (60 kDa), Ku, and PM/Scl75.

25. The composition of claim 23, wherein the at least one TAA comprises at least one breast cancer TAA.

26. The composition of claim 23, wherein the at least one TAA is selected from the group consisting of ANGPTL4, CALD1, CD147, CD320, CDH3, CST2, DKK1, EPHA2, GAL1, GFRA1, GRN, Her2, IGFBP2, LAMC2, LRP10, LRRC15, MUC1, SPINT2, SPON2, SSR2, SUSD2, XAGE-1B, A1AT, BIRC5, GAL3, CAPC, and GRP78.

27. The composition of claim 23, comprising second antibody detection marker molecules that bind to human autoantibodies against at least 2, 3, 4, 5, 6, 7, 8, 9, 10, or more ENAs.

28. The composition of claim 23, comprising first antibody detection marker molecules that bind to human autoantibodies against at least 2, 3, 4, 5, 6, 7, 8, 9, 10, or more TAAs.

29. The composition of claim 23, wherein the at least one ENA includes 1, 2, 3, or all 4 of SS-A/Ro (52 kDa), SS-B/La, CENPB, and Ku.

30. The composition of claim 23, wherein the at least one TAA includes 1, 2, 3, 4, 5, 6, 7, 8, 9, 10, or all 11 of ANGPTL4, CALD1, CD320, CDH3, DKK1, LRRC15, MUC1, SPINT2, SPON2, SSR2, and GRP78.

31. The composition of claim 23, wherein the combination of one or more TAA and one or more ENA comprises a set selected from the group consisting of:
A1AT and Sm;
A1AT and IGFBP2;
A1AT and GRN;
A1AT and CENPA;
A1AT, Sm, and IGFPB2;
A1AT, Sm, and GRN;
A1AT, Sm, and CENPA;
A1AT, Sm, IGFBP2, and GRN;
A1AT, Sm, IGFBP2, and CENPA
A1AT, Sm, IGFBP2, GRN, and CENPA;
A1AT, IGFBP2, and GRN;
A1AT, IGFBP2, and CENPA;
A1AT, IGFBP2, GRN, and CENPA;
Sm and IGFPB2;
Sm and GRN;
Sm and CENPA;
Sm, IGFPB2, and GRN;
Sm, IGFPB2, and CENPA;
Sm, IGFPB2, GRN, and CENPA;
IGFPB2 and GRN;
IGFPB2 and CENPA;
IGFPB2, GRN, and CENPA;
GRN and CENPA;
DKK1 and SS-A/Ro;
DKKI and SS-B/La;
DKKI and Ku;
DKKI, SS-A/Ro, and SS-B/La;
DKK1, SS-A/Ro, and Ku;
DKK1, SS-B/La, and Ku;
DKK1, ANGPTL4, and SS-A/Ro;
DKKI, ANGPTL4, and SS-B/La;
DKKI, ANGPTL4, and Ku;
DKKI, ANGPTL4, SS-A/Ro, and SS-B/La;
DKK1, ANGPTL4, SS-A/Ro, and Ku;
DKK1, ANGPTL4, SS-B/La, and Ku;
DKK1, MUC1, and SS-A/Ro;
DKKI, MUC1, and SS-B/La;
DKKI, MUC1, and Ku;
DKKI, MUC1, SS-A/Ro, and SS-B/La;
DKK1, MUC1, SS-A/Ro, and Ku;
DKK1, MUC1, SS-B/La, and Ku;
DKK1, ANGPTL4, MUC1, and SS-A/Ro;
DKKI, ANGPTL4, MUC1, and SS-B/La;
DKKI, ANGPTL4, MUC1, and Ku;
DKKI, ANGPTL4, MUC1, SS-A/Ro, and SS-B/La;
DKK1, ANGPTL4, MUC1, SS-A/Ro, and Ku;
DKK1, ANGPTL4, MUC1, SS-B/La, and Ku;
DKK1, GRP78, and SS-A/Ro;
DKKI, GRP78, and SS-B/La;
DKKI, GRP78, and Ku;
DKKI, GRP78, SS-A/Ro, and SS-B/La;
DKK1, GRP78, SS-A/Ro, and Ku;
DKK1, GRP78, SS-B/La, and Ku;
DKK1, ANGPTL4, GRP78, and SS-A/Ro;
DKKI, ANGPTL4, GRP78, and SS-B/La;
DKKI, ANGPTL4, GRP78, and Ku;
DKKI, ANGPTL4, GRP78, SS-A/Ro, and SS-B/La;
DKK1, ANGPTL4, GRP78, SS-A/Ro, and Ku;
DKK1, ANGPTL4, GRP78, SS-B/La, and Ku;
DKK1, MUC1, GRP78, and SS-A/Ro;
DKKI, MUC1, GRP78, and SS-B/La;
DKKI, MUC1, GRP78, and Ku;
DKKI, MUC1, GRP78, SS-A/Ro, and SS-B/La;
DKK1, MUC1, GRP78, SS-A/Ro, and Ku;
DKK1, MUC1, GRP78, SS-B/La, and Ku;
DKK1, ANGPTL4, MUC1, GRP78, and SS-A/Ro;
DKKI, ANGPTL4, MUC1, GRP78, and SS-B/La;
DKKI, ANGPTL4, MUC1, GRP78, and Ku;
DKKI, ANGPTL4, MUC1, GRP78, SS-A/Ro, and SS-B/La;
DKK1, ANGPTL4, MUC1, GRP78, SS-A/Ro, and Ku;
DKK1, ANGPTL4, MUC1, GRP78, SS-B/La, and Ku;
DKK1, GFRA1, and SS-A/Ro;
DKKI, GFRA1, and SS-B/La;
DKKI, GFRA1, and Ku;
DKKI, GFRA1, SS-A/Ro, and SS-B/La;
DKK1, GFRA1, SS-A/Ro, and Ku;
DKK1, GFRA1, SS-B/La, and Ku;
DKK1, ANGPTL4, GFRA1, and SS-A/Ro;
DKKI, ANGPTL4, GFRA1, and SS-B/La;
DKKI, ANGPTL4, GFRA1, and Ku;
DKKI, ANGPTL4, GFRA1, SS-A/Ro, and SS-B/La;
DKK1, ANGPTL4, GFRA1, SS-A/Ro, and Ku;
DKK1, ANGPTL4, GFRA1, SS-B/La, and Ku;
DKK1, MUC1, GFRA1, and SS-A/Ro;
DKKI, MUC1, GFRA1, and SS-B/La;
DKKI, MUC1, GFRA1, and Ku;
DKKI, MUC1, GFRA1, SS-A/Ro, and SS-B/La;
DKK1, MUC1, GFRA1, SS-A/Ro, and Ku;
DKK1, MUC1, GFRA1, SS-B/La, and Ku;
DKK1, ANGPTL4, MUC1, GFRA1, and SS-A/Ro;
DKKI, ANGPTL4, MUC1, GFRA1, and SS-B/La;
DKKI, ANGPTL4, MUC1, GFRA1, and Ku;
DKKI, ANGPTL4, MUC1, GFRA1, SS-A/Ro, and SS-B/La;
DKK1, ANGPTL4, MUC1, GFRA1, SS-A/Ro, and Ku;
DKK1, ANGPTL4, MUC1, GFRA1, SS-B/La, and Ku;
DKK1, ANGPTL4, MUC1, GRP78, GFRA1, and SS-A/Ro;
DKKI, ANGPTL4, MUC1, GRP78, GFRA1, and SS-B/La;
DKKI, ANGPTL4, MUC1, GRP78, GFRA1, and Ku;
DKKI, ANGPTL4, MUC1, GRP78, GFRA1, SS-A/Ro, and SS-B/La;
DKK1, ANGPTL4, MUC1, GRP78, GFRA1, SS-A/Ro, and Ku;
DKK1, ANGPTL4, MUC1, GRP78, GFRA1, SS-B/La, and Ku;
DKK1, A1AT, and SS-A/Ro;
DKKI, A1AT, and SS-B/La;
DKKI, A1AT, and Ku;
DKKI, A1AT, SS-A/Ro, and SS-B/La;
DKK1, A1AT, SS-A/Ro, and Ku;
DKK1, A1AT, SS-B/La, and Ku;
DKK1, ANGPTL4, A1AT, and SS-A/Ro;
DKKI, ANGPTL4, A1AT, and SS-B/La;
DKKI, ANGPTL4, A1AT, and Ku;
DKKI, ANGPTL4, A1AT, SS-A/Ro, and SS-B/La;
DKK1, ANGPTL4, A1AT, SS-A/Ro, and Ku;
DKK1, ANGPTL4, A1AT, SS-B/La, and Ku;
DKK1, MUC1, A1AT, and SS-A/Ro;
DKKI, MUC1, A1AT, and SS-B/La;
DKKI, MUC1, A1AT, and Ku;
DKKI, MUC1, A1AT, SS-A/Ro, and SS-B/La;
DKK1, MUC1, A1AT, SS-A/Ro, and Ku;
DKK1, MUC1, A1AT, SS-B/La, and Ku;
DKK1, ANGPTL4, MUC1, A1AT, and SS-A/Ro;
DKKI, ANGPTL4, MUC1, A1AT, and SS-B/La;
DKKI, ANGPTL4, MUC1, A1AT, and Ku;
DKKI, ANGPTL4, MUC1, A1AT, SS-A/Ro, and SS-B/La;
DKK1, ANGPTL4, MUC1, A1AT, SS-A/Ro, and Ku;
DKK1, ANGPTL4, MUC1, A1AT, SS-B/La, and Ku;
DKK1, ANGPTL4, MUC1, GRP78, A1AT, and SS-A/Ro;
DKKI, ANGPTL4, MUC1, GRP78, A1AT, and SS-B/La;
DKKI, ANGPTL4, MUC1, GRP78, A1AT, and Ku;
DKKI, ANGPTL4, MUC1, GRP78, A1AT, SS-A/Ro, and SS-B/La;
DKK1, ANGPTL4, MUC1, GRP78, A1AT, SS-A/Ro, and Ku;
DKK1, ANGPTL4, MUC1, GRP78, A1AT, SS-B/La, and Ku;
DKK1, ANGPTL4, MUC1, GRP78, GFRA1, A1AT, and SS-A/Ro;
DKKI, ANGPTL4, MUC1, GRP78, GFRA1, A1AT, and SS-B/La;
DKKI, ANGPTL4, MUC1, GRP78, GFRA1, A1AT, and Ku;
DKKI, ANGPTL4, MUC1, GRP78, GFRA1, A1AT, SS-A/Ro, and SS-B/La;
DKK1, ANGPTL4, MUC1, GRP78, GFRA1, A1AT, SS-A/Ro, and Ku;
DKK1, ANGPTL4, MUC1, GRP78, GFRA1, A1AT, SS-B/La, and Ku;
DKK1, GRN, and SS-A/Ro;
DKKI, GRN, and SS-B/La;
DKKI, GRN, and Ku;
DKKI, GRN, SS-A/Ro, and SS-B/La;
DKK1, GRN, SS-A/Ro, and Ku;
DKK1, GRN, SS-B/La, and Ku;
DKK1, ANGPTL4, GRN, and SS-A/Ro;
DKKI, ANGPTL4, GRN, and SS-B/La;
DKKI, ANGPTL4, GRN, and Ku;
DKKI, ANGPTL4, GRN, SS-A/Ro, and SS-B/La;
DKK1, ANGPTL4, GRN, SS-A/Ro, and Ku;
DKK1, ANGPTL4, GRN, SS-B/La, and Ku;
DKK1, MUC1, GRN, and SS-A/Ro;
DKKI, MUC1, GRN, and SS-B/La;
DKKI, MUC1, GRN, and Ku;
DKKI, MUC1, GRN, SS-A/Ro, and SS-B/La;
DKK1, MUC1, GRN, SS-A/Ro, and Ku;
DKK1, MUC1, GRN, SS-B/La, and Ku;
DKK1, ANGPTL4, MUC1, GRN, and SS-A/Ro;
DKKI, ANGPTL4, MUC1, GRN, and SS-B/La;
DKKI, ANGPTL4, MUC1, GRN, and Ku;
DKKI, ANGPTL4, MUC1, GRN, SS-A/Ro, and SS-B/La;
DKK1, ANGPTL4, MUC1, GRN, SS-A/Ro, and Ku;
DKK1, ANGPTL4, MUC1, GRN, SS-B/La, and Ku;
DKK1, ANGPTL4, MUC1, GRP78, GRN, and SS-A/Ro;
DKKI, ANGPTL4, MUC1, GRP78, GRN, and SS-B/La;
DKKI, ANGPTL4, MUC1, GRP78, GRN, and Ku;
DKKI, ANGPTL4, MUC1, GRP78, GRN, SS-A/Ro, and SS-B/La;
DKK1, ANGPTL4, MUC1, GRP78, GRN, SS-A/Ro, and Ku;
DKK1, ANGPTL4, MUC1, GRP78, GRN, SS-B/La, and Ku;
DKK1, ANGPTL4, MUC1, GRP78, GFRA1, GRN, and SS-A/Ro;
DKKI, ANGPTL4, MUC1, GRP78, GFRA1, GRN, and SS-B/La;
DKKI, ANGPTL4, MUC1, GRP78, GFRA1, GRN, and Ku;
DKKI, ANGPTL4, MUC1, GRP78, GFRA1, GRN, SS-A/Ro, and SS-B/La;
DKK1, ANGPTL4, MUC1, GRP78, GFRA1, GRN, SS-A/Ro, and Ku;
DKK1, ANGPTL4, MUC1, GRP78, GFRA1, GRN, SS-B/La, and Ku;
DKK1, ANGPTL4, MUC1, GRP78, GFRA1, A1AT, GRN, and SS-A/Ro;
DKKI, ANGPTL4, MUC1, GRP78, GFRA1, A1AT, GRN, and SS-B/La;
DKKI, ANGPTL4, MUC1, GRP78, GFRA1, A1AT, GRN, and Ku;
DKKI, ANGPTL4, MUC1, GRP78, GFRA1, A1AT, GRN, SS-A/Ro, and SS-B/La;
DKK1, ANGPTL4, MUC1, GRP78, GFRA1, A1AT, GRN, SS-A/Ro, and Ku;
DKK1, ANGPTL4, MUC1, GRP78, GFRA1, A1AT, GRN, SS-B/La, and Ku;
ANGPTL4 and SS-A/Ro;
ANGPTL4 and SS-B/La;
ANGPTL4 and Ku;
ANGPTL4, SS-A/Ro, and SS-B/La;
ANGPTL4, SS-A/Ro, and Ku;
ANGPTL4, SS-B/La, and Ku;
ANGPTL4, MUC1, and SS-A/Ro;
ANGPTL4, MUC1, and SS-B/La;
ANGPTL4, MUC1, and Ku;
ANGPTL4, MUC1, SS-A/Ro, and SS-B/La;
ANGPTL4, MUC1, SS-A/Ro, and Ku;
ANGPTL4, MUC1, SS-B/La, and Ku;
ANGPTL4, GRP78, and SS-A/Ro;
ANGPTL4, GRP78, and SS-B/La;
ANGPTL4, GRP78, and Ku;
ANGPTL4, GRP78, SS-A/Ro, and SS-B/La;
ANGPTL4, GRP78, SS-A/Ro, and Ku;
ANGPTL4, GRP78, SS-B/La, and Ku;
ANGPTL4, MUC1, GRP78, and SS-A/Ro;
ANGPTL4, MUC1, GRP78, and SS-B/La;
ANGPTL4, MUC1, GRP78, and Ku;
ANGPTL4, MUC1, GRP78, SS-A/Ro, and SS-B/La;
ANGPTL4, MUC1, GRP78, SS-A/Ro, and Ku;
ANGPTL4, MUC1, GRP78, SS-B/La, and Ku;
ANGPTL4, GFRA1, and SS-A/Ro;
ANGPTL4, GFRA1, and SS-B/La;
ANGPTL4, GFRA1, and Ku;
ANGPTL4, GFRA1, SS-A/Ro, and SS-B/La;
ANGPTL4, GFRA1, SS-A/Ro, and Ku;
ANGPTL4, GFRA1, SS-B/La, and Ku;
ANGPTL4, MUC1, GFRA1, and SS-A/Ro;
ANGPTL4, MUC1, GFRA1, and SS-B/La;
ANGPTL4, MUC1, GFRA1, and Ku;
ANGPTL4, MUC1, GFRA1, SS-A/Ro, and SS-B/La;
ANGPTL4, MUC1, GFRA1, SS-A/Ro, and Ku;
ANGPTL4, MUC1, GFRA1, SS-B/La, and Ku;
ANGPTL4, MUC1, GRP78, GFRA1, and SS-A/Ro;
ANGPTL4, MUC1, GRP78, GFRA1, and SS-B/La;
ANGPTL4, MUC1, GRP78, GFRA1, and Ku;
ANGPTL4, MUC1, GRP78, GFRA1, SS-A/Ro, and SS-B/La;
ANGPTL4, MUC1, GRP78, GFRA1, SS-A/Ro, and Ku;
ANGPTL4, MUC1, GRP78, GFRA1, SS-B/La, and Ku;
ANGPTL4, A1AT, and SS-A/Ro;
ANGPTL4, A1AT, and SS-B/La;
ANGPTL4, A1AT, and Ku;
ANGPTL4, A1AT, SS-A/Ro, and SS-B/La;
ANGPTL4, A1AT, SS-A/Ro, and Ku;
ANGPTL4, A1AT, SS-B/La, and Ku;
ANGPTL4, MUC1, A1AT, and SS-A/Ro;
ANGPTL4, MUC1, A1AT, and SS-B/La;
ANGPTL4, MUC1, A1AT, and Ku;
ANGPTL4, MUC1, A1AT, SS-A/Ro, and SS-B/La;
ANGPTL4, MUC1, A1AT, SS-A/Ro, and Ku;
ANGPTL4, MUC1, A1AT, SS-B/La, and Ku;
ANGPTL4, MUC1, GRP78, A1AT, and SS-A/Ro;
ANGPTL4, MUC1, GRP78, A1AT, and SS-B/La;
ANGPTL4, MUC1, GRP78, A1AT, and Ku;
ANGPTL4, MUC1, GRP78, A1AT, SS-A/Ro, and SS-B/La;
ANGPTL4, MUC1, GRP78, A1AT, SS-A/Ro, and Ku;
ANGPTL4, MUC1, GRP78, A1AT, SS-B/La, and Ku;
ANGPTL4, MUC1, GRP78, GFRA1, A1AT, and SS-A/Ro;
ANGPTL4, MUC1, GRP78, GFRA1, A1AT, and SS-B/La;
ANGPTL4, MUC1, GRP78, GFRA1, A1AT, and Ku;
ANGPTL4, MUC1, GRP78, GFRA1, A1AT, SS-A/Ro, and SS-B/La;
ANGPTL4, MUC1, GRP78, GFRA1, A1AT, SS-A/Ro, and Ku;
ANGPTL4, MUC1, GRP78, GFRA1, A1AT, SS-B/La, and Ku;
ANGPTL4, GRN, and SS-A/Ro;
ANGPTL4, GRN, and SS-B/La;
ANGPTL4, GRN, and Ku;
ANGPTL4, GRN, SS-A/Ro, and SS-B/La;
ANGPTL4, GRN, SS-A/Ro, and Ku;
ANGPTL4, GRN, SS-B/La, and Ku;
ANGPTL4, MUC1, GRN, and SS-A/Ro;
ANGPTL4, MUC1, GRN, and SS-B/La;
ANGPTL4, MUC1, GRN, and Ku;
ANGPTL4, MUC1, GRN, SS-A/Ro, and SS-B/La;
ANGPTL4, MUC1, GRN, SS-A/Ro, and Ku;
ANGPTL4, MUC1, GRN, SS-B/La, and Ku;
ANGPTL4, MUC1, GRP78, GRN, and SS-A/Ro;
ANGPTL4, MUC1, GRP78, GRN, and SS-B/La;
ANGPTL4, MUC1, GRP78, GRN, and Ku;
ANGPTL4, MUC1, GRP78, GRN, SS-A/Ro, and SS-B/La;
ANGPTL4, MUC1, GRP78, GRN, SS-A/Ro, and Ku;
ANGPTL4, MUC1, GRP78, GRN, SS-B/La, and Ku;
ANGPTL4, MUC1, GRP78, GFRA1, GRN, and SS-A/Ro;
ANGPTL4, MUC1, GRP78, GFRA1, GRN, and SS-B/La;
ANGPTL4, MUC1, GRP78, GFRA1, GRN, and Ku;
ANGPTL4, MUC1, GRP78, GFRA1, GRN, SS-A/Ro, and SS-B/La;
ANGPTL4, MUC1, GRP78, GFRA1, GRN, SS-A/Ro, and Ku;
ANGPTL4, MUC1, GRP78, GFRA1, GRN, SS-B/La, and Ku;
ANGPTL4, MUC1, GRP78, GFRA1, A1AT, GRN, and SS-A/Ro;
ANGPTL4, MUC1, GRP78, GFRA1, A1AT, GRN, and SS-B/La;
ANGPTL4, MUC1, GRP78, GFRA1, A1AT, GRN, and Ku;
ANGPTL4, MUC1, GRP78, GFRA1, A1AT, GRN, SS-A/Ro, and SS-B/La;
ANGPTL4, MUC1, GRP78, GFRA1, A1AT, GRN, SS-A/Ro, and Ku;
ANGPTL4, MUC1, GRP78, GFRA1, A1AT, GRN, SS-B/La, and Ku;
MUC1 and SS-A/Ro;
MUC1 and SS-B/La;
MUC1 and Ku;
MUC1, SS-A/Ro, and SS-B/La;
MUC1, SS-A/Ro, and Ku;
MUC1, SS-B/La, and Ku;
MUC1, GRP78, and SS-A/Ro;
MUC1, GRP78, and SS-B/La;
MUC1, GRP78, and Ku;
MUC1, GRP78, SS-A/Ro, and SS-B/La;
MUC1, GRP78, SS-A/Ro, and Ku;
MUC1, GRP78, SS-B/La, and Ku;
MUC1, GFRA1, and SS-A/Ro;
MUC1, GFRA1, and SS-B/La;
MUC1, GFRA1, and Ku;
MUC1, GFRA1, SS-A/Ro, and SS-B/La;
MUC1, GFRA1, SS-A/Ro, and Ku;
MUC1, GFRA1, SS-B/La, and Ku;
MUC1, GRP78, GFRA1, and SS-A/Ro;
MUC1, GRP78, GFRA1, and SS-B/La;
MUC1, GRP78, GFRA1, and Ku;
MUC1, GRP78, GFRA1, SS-A/Ro, and SS-B/La;
MUC1, GRP78, GFRA1, SS-A/Ro, and Ku;
MUC1, GRP78, GFRA1, SS-B/La, and Ku;
MUC1, A1AT, and SS-A/Ro;
MUC1, A1AT, and SS-B/La;
MUC1, A1AT, and Ku;
MUC1, A1AT, SS-A/Ro, and SS-B/La;
MUC1, A1AT, SS-A/Ro, and Ku;
MUC1, A1AT, SS-B/La, and Ku;
MUC1, GRP78, A1AT, and SS-A/Ro;
MUC1, GRP78, A1AT, and SS-B/La;
MUC1, GRP78, A1AT, and Ku;
MUC1, GRP78, A1AT, SS-A/Ro, and SS-B/La;
MUC1, GRP78, A1AT, SS-A/Ro, and Ku;
MUC1, GRP78, A1AT, SS-B/La, and Ku;
MUC1, GRP78, GFRA1, A1AT, and SS-A/Ro;
MUC1, GRP78, GFRA1, A1AT, and SS-B/La;
MUC1, GRP78, GFRA1, A1AT, and Ku;
MUC1, GRP78, GFRA1, A1AT, SS-A/Ro, and SS-B/La;
MUC1, GRP78, GFRA1, A1AT, SS-A/Ro, and Ku;
MUC1, GRP78, GFRA1, A1AT, SS-B/La, and Ku;
MUC1, GRN, and SS-A/Ro;
MUC1, GRN, and SS-B/La;
MUC1, GRN, and Ku;
MUC1, GRN, SS-A/Ro, and SS-B/La;
MUC1, GRN, SS-A/Ro, and Ku;
MUC1, GRN, SS-B/La, and Ku;
MUC1, GRP78, GRN, and SS-A/Ro;
MUC1, GRP78, GRN, and SS-B/La;
MUC1, GRP78, GRN, and Ku;
MUC1, GRP78, GRN, SS-A/Ro, and SS-B/La;
MUC1, GRP78, GRN, SS-A/Ro, and Ku;
MUC1, GRP78, GRN, SS-B/La, and Ku;
MUC1, GRP78, GFRA1, GRN, and SS-A/Ro;
MUC1, GRP78, GFRA1, GRN, and SS-B/La;
MUC1, GRP78, GFRA1, GRN, and Ku;
MUC1, GRP78, GFRA1, GRN, SS-A/Ro, and SS-B/La;
MUC1, GRP78, GFRA1, GRN, SS-A/Ro, and Ku;
MUC1, GRP78, GFRA1, GRN, SS-B/La, and Ku;
MUC1, GRP78, GFRA1, A1AT, GRN, and SS-A/Ro;
MUC1, GRP78, GFRA1, A1AT, GRN, and SS-B/La;
MUC1, GRP78, GFRA1, A1AT, GRN, and Ku;
MUC1, GRP78, GFRA1, A1AT, GRN, SS-A/Ro, and SS-B/La;
MUC1, GRP78, GFRA1, A1AT, GRN, SS-A/Ro, and Ku;
MUC1, GRP78, GFRA1, A1AT, GRN, SS-B/La, and Ku;
GRP78 and SS-A/Ro;
GRP78 and SS-B/La;
GRP78 and Ku;
GRP78, SS-A/Ro, and SS-B/La;
GRP78, SS-A/Ro, and Ku;
GRP78, SS-B/La, and Ku;
GRP78, GFRA1, and SS-A/Ro;
GRP78, GFRA1, and SS-B/La;
GRP78, GFRA1, and Ku;
GRP78, GFRA1, SS-A/Ro, and SS-B/La;
GRP78, GFRA1, SS-A/Ro, and Ku;
GRP78, GFRA1, SS-B/La, and Ku;
GRP78, A1AT, and SS-A/Ro;
GRP78, A1AT, and SS-B/La;
GRP78, A1AT, and Ku;
GRP78, A1AT, SS-A/Ro, and SS-B/La;
GRP78, A1AT, SS-A/Ro, and Ku;
GRP78, A1AT, SS-B/La, and Ku;
GRP78, GFRA1, A1AT, and SS-A/Ro;
GRP78, GFRA1, A1AT, and SS-B/La;
GRP78, GFRA1, A1AT, and Ku;
GRP78, GFRA1, A1AT, SS-A/Ro, and SS-B/La;
GRP78, GFRA1, A1AT, SS-A/Ro, and Ku;
GRP78, GFRA1, A1AT, SS-B/La, and Ku;
GRP78, GRN, and SS-A/Ro;
GRP78, GRN, and SS-B/La;
GRP78, GRN, and Ku;
GRP78, GRN, SS-A/Ro, and SS-B/La;
GRP78, GRN, SS-A/Ro, and Ku;
GRP78, GRN, SS-B/La, and Ku;
GRP78, GFRA1, GRN, and SS-A/Ro;
GRP78, GFRA1, GRN, and SS-B/La;
GRP78, GFRA1, GRN, and Ku;
GRP78, GFRA1, GRN, SS-A/Ro, and SS-B/La;
GRP78, GFRA1, GRN, SS-A/Ro, and Ku;
GRP78, GFRA1, GRN, SS-B/La, and Ku;
GRP78, GFRA1, A1AT, GRN, and SS-A/Ro;
GRP78, GFRA1, A1AT, GRN, and SS-B/La;
GRP78, GFRA1, A1AT, GRN, and Ku;
GRP78, GFRA1, A1AT, GRN, SS-A/Ro, and SS-B/La;
GRP78, GFRA1, A1AT, GRN, SS-A/Ro, and Ku;
GRP78, GFRA1, A1AT, GRN, SS-B/La, and Ku;
GFRA1 and SS-A/Ro;
GFRA1 and SS-B/La;
GFRA1 and Ku;
GFRA1, SS-A/Ro, and SS-B/La;
GFRA1, SS-A/Ro, and Ku;
GFRA1, SS-B/La, and Ku;
GFRA1, A1AT, and SS-A/Ro;
GFRA1, A1AT, and SS-B/La;
GFRA1, A1AT, and Ku;
GFRA1, A1AT, SS-A/Ro, and SS-B/La;
GFRA1, A1AT, SS-A/Ro, and Ku;
GFRA1, A1AT, SS-B/La, and Ku;
GFRA1, GRN, and SS-A/Ro;
GFRA1, GRN, and SS-B/La;
GFRA1, GRN, and Ku;
GFRA1, GRN, SS-A/Ro, and SS-B/La;
GFRA1, GRN, SS-A/Ro, and Ku;
GFRA1, GRN, SS-B/La, and Ku;
GFRA1, A1AT, GRN, and SS-A/Ro;
GFRA1, A1AT, GRN, and SS-B/La;
GFRA1, A1AT, GRN, and Ku;
GFRA1, A1AT, GRN, SS-A/Ro, and SS-B/La;
GFRA1, A1AT, GRN, SS-A/Ro, and Ku;
GFRA1, A1AT, GRN, SS-B/La, and Ku;
A1AT and SS-A/Ro;
A1AT and SS-B/La;
A1AT and Ku;
A1AT, SS-A/Ro, and SS-B/La;
A1AT, SS-A/Ro, and Ku;
A1AT, SS-B/La, and Ku;
A1AT, GRN, and SS-A/Ro;
A1AT, GRN, and SS-B/La;
A1AT, GRN, and Ku;
A1AT, GRN, SS-A/Ro, and SS-B/La;
A1AT, GRN, SS-A/Ro, and Ku;
A1AT, GRN, SS-B/La, and Ku

32. The composition of claim 23, wherein the one or more first antibody detection marker molecules comprise or consist of the TAA, an antigenic fragment thereof, or a native extracellular domain and/or native secreted version of the TAA or antigenic fragment thereof; and the one or more second antibody detection marker molecules comprise or consist of the ENA or antigenic fragment thereof, or a native extracellular domains and/or native secreted version of the ENA or antigenic fragment thereof.

33. The composition of claim 23, wherein the composition consists of between 2-10,000 antibody detection marker molecules in total.

34. The composition of claim 23, wherein the composition consists of between 2 and 1000 antibody detection marker molecules in total.


SEQUENCE LISTING (47 sequences)

SEQ ID NO: 1 - protein, 140 aa, Homo sapiens
MGPRRRSRKPEAPRRRSPSPTPTPGPSRRGPSLGASSHQHSRRRQGWLKEIRKLQKSTHLLIRKLPFSRLAREICVKFTRGVDFNWQAQALLALQEAAEAFLVHLFEDAYLLTLHAGRVTLFPKDVQLARRIRGLEEGLG

SEQ ID NO: 2 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 3 - protein, 437 aa, Homo sapiens
MTQFLPPNLLALFAPRDPIPYLPPLEKLPHEKHHNQPYCGIAPYIREFEDPRDAPPPTRAETREERMERKRREKIERRQQEVETELKMWDPHNDPNAQGDAFKTLFVARVNYDTTESKLRREFEVYGPIKRIHMVYSKRSGKPRGYAFIEYEHERDMHSAYKHADGKKIDGRRVLVDVERGRTVKGWRPRRLGGGLGGTRRGGADVNIRHSGRDDTSRYDERPGPSPLPHRDRDRDRERERRERSRERDKERERRRSRSRDRRRRSRSRDKEERRRSRERSKDKDRDRKRRSSRSRERARRERERKEELRGGGGDMAEPSEAGDAPPDDGPPGELGPDGPDGPEEKGRDRDRERRRSHRSERERRRDRDRDRDRDREHKRGERGSERGRDEARGGGGGQDNGLEGLGNDSRDMYMESEGGDGYLAPENGYLMEAAPE

SEQ ID NO: 4 - protein, 282 aa, Homo sapiens
MAVPETRPNHTIYINNLNEKIKKDELKKSLYAIFSQFGQILDILVSRSLKMRGQAFVIFKEVSSATNALRSMQGFPFYDKPMRIQYAKTDSDIIAKMKGTFVERDRKREKRKPKSQETPATKKAVQGGGATPVVGAVQGPVPGMPPMTQAPRIMHHMPGQPPYMPPPGMIPPPGLAPGQIPPGAMPPQQLMPGQMPPAQPLSENPPNHILFLTNLPEETNELMLSMLFNQFPGFKEVRLVPGRHDIAFVEFDNEVQAGAARDALQGFKITQNNAMKISFAKK

SEQ ID NO: 5 - protein, 159 aa, Homo sapiens
MPKFYCDYCDTYLTHDSPSVRKTHCSGRKHKENVKDYYQKWMEEQAQSLIDKTTAAFQQGKIPPTPFSAPPPAGAMIPPPPSLPGPPRPGMMPAPHMGGPPMMPMMGPPPPGMMPVGPAPGMRPPMGGHMPMMPGPPMMRPPARPMMVPTRPGMTRPDR

SEQ ID NO: 6 - protein, 765 aa, Homo sapiens
MSGDHLHNDSQIEADFRLNDSHKHKDKHKDREHRHKEHKKEKDREKSKHSNSEHKDSEKKHKEKEKTKHKDGSSEKHKDKHKDRDKEKRKEEKVRASGDAKIKKEKENGFSSPPQIKDEPEDDGYFVPPKEDIKPLKRPRDEDDADYKPKKIKTEDTKKEKKRKLEEEEDGKLKKPKNKDKDKKVPEPDNKKKKPKKEEEQKWKWWEEERYPEGIKWKFLEHKGPVFAPPYEPLPENVKFYYDGKVMKLSPKAEEVATFFAKMLDHEYTTKEIFRKNFFKDWRKEMTNEEKNIITNLSKCDFTQMSQYFKAQTEARKQMSKEEKLKIKEENEKLLKEYGFCIMDNHKERIANFKIEPPGLFRGRGNHPKMGMLKRRIMPEDIIINCSKDAKVPSPPPGHKWKEVRHDNKVTWLVSWTENIQGSIKYIMLNPSSRIKGEKDWQKYETARRLKKCVDKIRNQYREDWKSKEMKVRQRAVALYFIDKLALRAGNEKEEGETADTVGCCSLRVEHINLHPELDGQEYVVEFDFLGKDSIRYYNKVPVEKRVFKNLQLFMENKQPEDDLFDRLNTGILNKHLQDLMEGLTAKVFRTYNASITLQQQLKELTAPDENIPAKILSYNRANRAVAILCNHQRAPPKTFEKSMMNLQTKIDAKKEQLADARRDLKSAKADAKVMKDAKTKKVVESKKKAVQRLEEQLMKLEVQATDREENKQIALGTSKLNYLDPRITVAWCKKWGVPIEKIYNKTQREKFAWAIDMADEDYEF

SEQ ID NO: 7 - protein, 231 aa, Homo sapiens
MTVGKSSKMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGLL

SEQ ID NO: 8 - protein, 240 aa, Homo sapiens
MTVGKSSKMLQHIDYRMRCILQDGRIFIGTFKAFDKHMNLILCDCDEFRKIKPKNSKQAEREEKRVLGLVLLRGENLVSMTVEGPPPKDTGIARVPLAGAAGGPGIGRAAGRGIPAGVPMPQAPAGLAGPVRGVGGPSQQVMTPQGRGTVAAAAAAATASIAGAPTQYPPGRGGPPPPMGRGAPPPGMMGPPPGMRPPMGPPMGIPPGRGTPMGMPPPGMRPPPPGMRGPPPPGMRPPRP

SEQ ID NO: 9 - protein, 119 aa, Homo sapiens
MKLVRFLMKLSHETVTIELKNGTQVHGTITGVDVSMNTHLKAVKMTLKNREPVQLETLSIRGNNIRYFILPDSLPLDTLLVDVEPKVKSKKREAVAGRGRGRGRGRGRGRGRGRGGPRR

SEQ ID NO: 10 - protein, 92 aa, Homo sapiens
MAYRGQGQKVQKVMVQPINLIFRYLQNRSRIQVWLYEQVNMRIEGCIIGFDEYMNLVLDDAEEIHSKTKSRKQLGRIMLKGDNITLLQSVSN

SEQ ID NO: 11 - protein, 86 aa, Homo sapiens
MSLPLNPKPFLNGLTGKPVMVKLKWGMEYKGYLVSVDGYMNMQLANTEEYIDGALSGHLGEVLIRCNNVLYIRGVEEEEEDGEMRE

SEQ ID NO: 12 - protein, 76 aa, Homo sapiens
MSKAHPPELKKFMDKKLSLKLNGGRHVQGILRGFDPFMNLVIDECVEMATSGQQNNIGMVVIRGNSIIMLEALERV

SEQ ID NO: 13 - protein, 475 aa, Homo sapiens [sequence omitted]

SEQ ID NO: 14 - protein, 408 aa, Homo sapiens
MAENGDNEKMAALEAKICHQIEYYFGDFNLPRDKFLKEQIKLDEGWVPLEIMIKFNRLNRLTTDFNVIVEALSKSKAELMEISEDKTKIRRSPSKPLPEVTDEYKNDVKNRSVYIKGFPTDATLDDIKEWLEDKGQVLNIQMRRTLHKAFKGSIFVVFDSIESAKKFVETPGQKYKETDLLILFKDDYFAKKNEERKQNKVEAKLRAKQEQEAKQKLEEDAEMKSLEEKIGCLLKFSGDLDDQTCREDLHILFSNHGEIKWIDFVRGAKEGIILFKEKAKEALGKAKDANNGNLQLRNKEVTWEVLEGEVEKEALKKIIEDQQESLNKWKSKGRRFKGKGKGNKAAQPGSGKGKVQFQGKKTKFASDDEHDEHDENGATGPVKRAREETDKEEPASKQQKTENGAGDQ

SEQ ID NO: 15 - protein, 599 aa, Homo sapiens
MGPKRRQLTFREKSRIIQEVEENPDLRKGEIARRFNIPPSTLSTILKNKRAILASERKYGVASTCRKTNKLSPYDKLEGLLIAWFQQIRAAGLPVKGIILKEKALRIAEELGMDDFTASNGWLDRFRRRHGVVSCSGVARARARNAAPRTPAAPASPAAVPSEGSGGSTTGWRAREEQPPSVAEGYASQDVFSATETSLWYDFLPDQAAGLCGGDGRPRQATQRLSVLLCANADGSEKLPPLVAGKSAKPRAGQAGLPCDYTANSKGGVTTQALAKYLKALDTRMAAESRRVLLLAGRLAAQSLDTSGLRHVQLAFFPPGTVHPLERGVVQQVKGHYRQAMLLKAMAALEGQDPSGLQLGLTEALHFVAAAWQAVEPSDIAACFREAGFGGGPNATITTSLKSEGEEEEEEEEEEEEEEGEGEEEEEEGEEEEEEGGEGEELGEEEEVEEEGDVDSDEEEEEDEESSSEGLEAEDWAQGVVEAGGSFGAYGAQEEAQCPTLHFLEGGEDSDSDSEEEDDEEEDDEDEDDDDDEEDGDEVPVPSFGEAMAYFAMVKRYLTSFPIDDRVQSHILHLEHDLVHVTRKNHARQAGVRGLGHQS

SEQ ID NO: 16 - protein, 885 aa, Homo sapiens
MAPPSTREPRVLSATSATKSDGEMVLPGFPDADSFVKFALGSVVAVTKASGGLPQFGDEYDFYRSFPGFQAFCETQGDRLLQCMSRVMQYHGCRSNIKDRSKVTELEDKFDLLVDANDVILERVGILLDEASGVNKNQQPVLPAGLQVPKTVVSSWNRKAAEYGKKAKSETFRLLHAKNIIRPQLKFREKIDNSNTPFLPKIFIKPNAQKPLPQALSKERRERPQDRPEDLDVPPALADFIHQQRTQQVEQDMFAHPYQYELNHFTPADAVLQKPQPQLYRPIEETPCHFISSLDELVELNEKLLNCQEFAVDLEHHSYRSFLGLTCLMQISTRTEDFIIDTLELRSDMYILNESLTDPAIVKVFHGADSDIEWLQKDFGLYVVNMFDTHQAARLLNLGRHSLDHLLKLYCNVDSNKQYQLADWRIRPLPEEMLSYARDDTHYLLYIYDKMRLEMWERGNGQPVQLQVVWQRSRDICLKKFIKPIFTDESYLELYRKQKKHLNTQQLTAFQLLFAWRDKTARREDESYGYVLPNHMMLKIAEELPKEPQGIIACCNPVPPLVRQQINEMHLLIQQAREMPLLKSEVAAGVKKSGPLPSAERLENVLFGPHDCSHAPPDGYPIIPTSGSVPVQKQASLFPDEKEDNLLGTTCLIATAVITLFNEPSAEDSKKGPLTVAQKKAQNIMESFENPFRMFLPSLGHRAPVSQAAKFDPSTKIYEISNRWKLAQVQVQKDSKEAVKKKAAEQTAAREQAKEACKAAAEQAISVRQQVVLENAAKKRERATSDPRTTEQKQEKKRLKISKKPKDPEPPEKEFTPYDYSQSDFKAFAGNSKSKVSSQFDPNKQTPSGKKCIAAKKIKQSVGNKSMSFPTGKSDRGFRYNWPQR

SEQ ID NO: 17 - protein, 525 aa, Homo sapiens
MEESVNQMQPLNEKQIANSQDGYVWQVTDMNRLHRFLCFGSEGGTYYIKEQKLGLENAEALIRLIEDGRGCEVIQEIKSFSQEGRTTKQEPMLFALAICSQCSDISTKQAAFKAVSEVCRIPTHLFTFIQFKKDLKESMKCGMWGRALRKAIADWYNEKGGMALALAVTKYKQRNGWSHKDLLRLSHLKPSSEGLAIVTKYITKGWKEVHELYKEKALSVETEKLLKYLEAVEKVKRTRDELEVIHLIEEHRLVREHLLTNHLKSKEVWKALLQEMPLTALLRNLGKMTANSVLEPGNSEVSLVCEKLCNEKLLKKARIHPFHILIALETYKTGHGLRGKLKWRPDEEILKALDAAFYKTFKTVEPTGKRFLLAVDVSASMNQRVLGSILNASTVAAAMCMVVTRTEKDSYVVAFSDEMVPCPVTTDMTLQQVLMAMSQIPAGGTDCSLPMIWAQKTNTPADVFIVFTDNETFAGGVHPAIALREYRKKMDIPAKLIVCGMTSNGFTIADPDDRALQNTLLNKSF

SEQ ID NO: 18 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 19 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 20 - protein, 456 aa, Homo sapiens
MKETPLSNCERRFLLRAIEEKKRLDGRQTYDYRNIRISFGTDYGCCIVELGKTRVLGQVSCELVSPKLNRATEGILFFNLELSQMAAPAFEPGRQSDLLVKLNRLMERCLRNSKCIDTESLCVVAGEKVWQIRVDLHLLNHDGNIIDAASIAAIVALCHFRRPDVSVQGDEVTLYTPEERDPVPLSIHHMPICVSFAFFQQGTYLLVDPNEREERVMDGLLVIAMNKHREICTIQSSGGIMLLKDQVLRCSKIAGVKVAEITELILKALENDQKVRKEGGKFGFAESIANQRITAFKMEKAPIDTSDVEEKAEEIIAEAEPPSEVVSTPVLWTPGTAQIGEGVENSWGDLEDSEKEDDEGGGDQAIILDGIKMDTGVEVSDIGSQELGFHHVGQTGLEFLTSDAPIILSDSEEEEMIILEPDKNPKKIRTQTTSAKQEKAPSKKPVKRRKKKRAAN

SEQ ID NO: 21 - protein, 376 aa, Homo sapiens
KSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 22 - protein, 238 aa, Homo sapiens
VSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 23 - protein, 441 aa, Homo sapiens
DRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISDVFQQVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 24 - protein, 576 aa, Homo sapiens
TRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 25 - protein, 424 aa, Homo sapiens
HPDRIIFPNHACEDPPAVLLEVQGTLQRPLVRDSRTSPANCTWLILGSKEQTVTIRFQKLHLACGSERLTLRSPLQPLISLCEAPPSPLQLPGGNVTITYSYAGARAPMGQGFLLSYSQDWLMCLQEEFQCLNHRCVSAVQRCDGVDACGDGSDEAGCSSDPFPGLTPRPVPSLPCNVTLEDFYGVFSSPGYTHLASVSHPQSCHWLLDPHDGRRLAVRFTALDLGFGDAVHVYDGPGPPESSRLLRSLTHFSNGKAVTVETLSGQAVVSYHTVAWSNGRGFNATYHVRGYCLPWDRPCGLGSGLGAGEGLGERCYSEAQRCDGSWDCADGTDEEDCPGCPPGHFPCGAAGTSGATACYLPADRCNYQTFCADGADERRCRHCQPGNFRCRDEKCVYETWVCDGQPDCADGSDEWDCSYVLPRK

SEQ ID NO: 26 - protein, 121 aa, Homo sapiens
WSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

SEQ ID NO: 27 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 28 - protein, 118 aa, Homo sapiens
LRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 29 - protein, 145 aa, Homo sapiens
APKPATVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPG

SEQ ID NO: 30 - protein, 636 aa, Homo sapiens
EEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 31 - protein, 509 aa, Homo sapiens
KEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNL

SEQ ID NO: 32 - protein, 1090 aa, Homo sapiens
TSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMGM

SEQ ID NO: 33 - protein, 305 aa, Homo sapiens
QPLGGESICSARAPAKYSITFTGKWSQTAFPKQYPLFRPPAQWSSLLGAAHSSDYSMWRKNQYVSNGLRDFAERGEAWALMKEIEAAGEALQSVHEVFSAPAVPSGTGQTSAELEVQRRHSLVSFVVRIVPSPDWFVGVDSLDLCDGDRWREQAALDLYPYDAGTDSGFTFSSPNFATIPQDTVTEITSSSPSHPANSFYYPRLKALPPIARVTLLRLRQSPRAFIPPAPVLPSRDNEIVDSASVPETPLDCEVSLWSSWGLCGGHCGRLGTKSRTRYVRVQPANNGSPCPELEEEAECVPDNCV

SEQ ID NO: 34 - protein, 129 aa, Homo sapiens
EEGARLLASKSLLNRYAVEGRDLTLQYNIYNVGSSAALDVELSDDSFPPEDFGIVSGMLNVKWDRIAPASNVSHTVVLRPLKAGYFNFTSATITYLAQEDGPVVIGSTSAPGQGGILAQREFDRRFSPH

SEQ ID NO: 35 - protein, 517 aa, Homo sapiens
YHGCPSECTCSRASQVECTGARIVAVPTPLPWNAMSLQILNTHITELNESPFLNISALIALRIEKNELSRITPGAFRNLGSLRYLSLANNKLQVLPIGLFQGLDSLESLLLSSNQLLQIQPAHFSQCSNLKELQLHGNHLEYIPDGAFDHLVGLTKLNLGKNSLTHISPRVFQHLGNLQVLRLYENRLTDIPMGTFDGLVNLQELALQQNQIGLLSPGLFHNNHNLQRLYLSNNHISQLPPSVFMQLPQLNRLTLFGNSLKELSPGIFGPMPNLRELWLYDNHISSLPDNVFSNLRQLQVLILSRNQISFISPGAFNGLTELRELSLHTNALQDLDGNVFRMLANLQNISLQNNRLRQLPGNIFANVNGLMAIQLQNNQLENLPLGIFDHLGKLCELRLYDNPWRCDSDILPLRNWLLLNQPRLGTDTVPVCFSPANVRGQSLIIINVNVAVPSVHVPEVPSYPETPWYPDTPSYPDTTSVSSTTELTSPVEDYTDLTTIQVTDDRSVWGMTQAQSG

SEQ ID NO: 36 - protein, 145 aa, Homo sapiens; identical to SEQ ID NO: 29

SEQ ID NO: 37 - protein, 141 aa, Homo sapiens
AAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSS

SEQ ID NO: 38 - protein, 184 aa, Homo sapiens
AGPSSGSCPPTKFQCRTSGLCVPLTWRCDRDLDCSDGSDEEECRIEPCTQKGQCPPPPGLPCPCTGVSDCSGGTDKKLRNCSRLACLAGELRCTLSDDCIPLTWRCDGHPDCPDSSDELGCGTNEILPEGDATTMGPPVTLESVTSLRNATTMGPPVTLESVPSVGNATSSSAGDQSGSPTAYG

SEQ ID NO: 39 - protein, 630 aa, Homo sapiens
EPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQMTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGG

SEQ ID NO: 40 - protein, 630 aa, Homo sapiens
TQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

SEQ ID NO: 41 - protein, 289 aa, Homo sapiens
EVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 42 - protein, 171 aa, Homo sapiens
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKV

SEQ ID NO: 43 - protein, 758 aa, Homo sapiens
QESCSMRCGALDGPCSCHPTCSGLGTCCLDFRDFCLEILPYSGSMMGGKDFVVRHFKMSSPTDASVICRFKDSIQTLGHVDSSGQVHCVSPLLYESGRIPFTVSLDNGHSFPRAGTWLAVHPNKVSMMEKSELVNETRWQYYGTANTSGNLSLTWHVKSLPTQTITIELWGYEETGMPYSQEWTAKWSYLYPLATHIPNSGSFTFTPKPAPPSYQRWRVGALRIIDSKNYAGQKDVQALWTNDHALAWHLSDDFREDPVAWARTQCQAWEELEDQLPNFLEELPDCPCTLTQARADSGRFFTDYGCDMEQGSVCTYHPGAVHCVRSVQASLRYGSGQQCCYTADGTQLLTADSSGGSTPDRGHDWGAPPFRTPPRVPSMSHWLYDVLSFYYCCLWAPDCPRYMQRRPSNDCRNYRPPRLASAFGDPHFVTFDGTNFTFNGRGEYVLLEAALTDLRVQARAQPGTMSNGTETRGTGLTAVAVQEGNSDVVEVRLANRTGGLEVLLNQEVLSFTEQSWMDLKGMFLSVAAGDRVSIMLASGAGLEVSVQGPFLSVSVLLPEKFLTHTHGLLGTLNNDPTDDFTLHSGRVLPPGTSPQELFLFGANWTVHNASSLLTYDSWFLVHNFLYQPKHDPTFEPLFPSETTLNPSLAQEAAKLCGDDHFCNFDVAATGSLSTGTATRVAHQLHQRRMQSLQPVVSCGWLAPPPNGQKEGNRYLAGSTIYFHCDNGYSLAGAETSTCQADGTWSSPTPKCQPGRSYA

SEQ ID NO: 44 - protein, 558 aa, Homo sapiens
MLGGSGSHGRRSLAALSQIAYQRNDDDEEEAARERRRRARQERLRQKQEEESLGQVTDQVEVNAQNSVPDEEAKTTTTNTQVEGDDEAAFLERLARREERRQKRLQEALERQKEFDPTITDASLSLPSRRMQNDTAENETTEKEEKSESRQERYEIEETETVTKSYQKNDWRDAEENKKEDKEKEEEEEEKPKRGSIGENQGEEKGTKVQAKREKLQEDKPTFKKEEIKDEKIKKDKEPKEEVKSFMDRKKGFTEVKSQNGEFMTHKLKHTENTFSRPGGRASVDTKEAEGAPQVEAGKRLEELRRRRGETESEEFEKLKQKQQEAALELEELKKKREERRKVLEEEEQRRKQEEADRKLREEEEKRRLKEEIERRRAEAAEKRQKMPEDGLSDDKKPFKCFTPKGSSLKIEERAEFLNKSVQKSSGVKSTHQAAIVSKIDSRLEQYTSAIEGTKSAKPTKPAASDLPVPAEGVRNIKSMWEKGNVFSSPTAAGTPNKETAGLKVGVSSRINEWLTKTPDGNKSPAPKPSDLRPGDVSSKRNLWEKQSVDKVTSPTKV

SEQ ID NO: 45 - protein, 81 aa, Homo sapiens
MESPKKKNQQLKVGILHLGSRQKKIRIQLRSQCATWKVICKSCISQTPGINLDLGSGVKVKIIPKEEHCKMPEAGEEQPQV

SEQ ID NO: 46 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMD

SEQ ID NO: 47 - protein, 250 aa, Homo sapiens [sequence omitted]